(19) United States
(12) Patent Application Publication
(10) Pub. No.: US 2021/0196674 A1
(43) Pub. Date: Jul. 1, 2021

(54) Therapeutics for Malaria

(71) Applicant: Jerry T. Thornthwaite, Henderson, TN (US)

(72) Inventor: Jerry T. Thornthwaite, Henderson, TN (US)

(21) Appl. No.: 17/197,001

(22) Filed: Mar. 9, 2021

Related U.S. Application Data
(63) Continuation of application No. 15/762,537, filed on Mar. 22, 2018, now abandoned.
(60) Provisional application No. 62/222,090, filed on Sep. 22, 2015.

(51) Int. Cl. A61K 31/357; A61K 31/365; A61K 31/366; A61K 36/282; A61K 9/127; A61P 33/06; A61K 45/06; A61K 47/10

(52) U.S. Cl. CPC A61K 31/357; A61K 31/365; A61K 31/366; A61K 36/282; Y02A 50/30; A61P 33/06; A61K 45/06; A61K 47/10; A61K 9/127

(57) ABSTRACT

The present disclosure describes a composition and method for treating and preventing malaria. The disclosure describes a peroxybioflavonoid composition comprising an artemisinin compound and one or more bioflavonoid compounds.

### CROSS-REFERENCE TO RELATED APPLICATIONS

This application is a continuation of U.S. patent application Ser. No. 15/762,537, filed Mar. 22, 2018, which is a 371 U.S. national stage entry of PCT Application Serial No. PCT/US2016/053154, filed Sep. 22, 2016, which claims the benefit of U.S. Provisional Patent Application Ser. No. 62/222,090, filed Sep. 22, 2015, each of which is incorporated herein by reference in its entirety.

### BACKGROUND

### Field of the Invention

The present invention relates generally to a method and formulation for a treating and/or preventing malaria treatment. More specifically, the present invention is the administration of low-dose Artemisinin, or derivatives of Artemisinin, with or without co-administration of bioflavonoids.

### Background

Malaria infection was first described at least as early as 4,000 years ago. In ancient China, India, Greece, Rome, and the Middle East, malaria and its possible treatments were documented. In ancient China, patients were given a treatment based on Artemisinin (documented 168 BC), an active ingredient in some current drug combinations.

Malaria is caused by infection with Plasmodium sp., a unicellular eukaryote. Among the five species of Plasmodium that infect humans, P. falciparum is the deadliest. Today, there are more than one million malaria-related deaths per year in endemic areas of sub-Saharan Africa, where 90% of all malaria-related deaths occur.

There is no effective preventative malaria vaccine, and therefore, control depends heavily on antimalarial drugs that kill parasites inside the human body. Many current therapies combine Artemisinin with other anti-malarial drugs. Many such Artemisinin-based combination therapies (“ACT”) are available and others continue to be developed. However, ACT therapies have a high production cost which limits their widespread application in major endemic areas. Additionally, parasitic resistance to the anti-malarial drugs present in these ACT therapies continues to develop. The continuing evolution of parasite resistance against affordable drugs results in an enormous cost for fighting the spread of the disease. Facing this reality, the focus of public health policy is being shifted to increasing the sustainability of Artemisinin treatment by delaying the emergence and spread of drug resistance as much as possible.

1. Development of Resistance to Anti-Malarial Drugs

One of the most challenging problems in the effective treatment of malaria in endemic regions is the development of drug resistant parasites. For example, Sulfadoxine-pyrimethamine (SP), a combination of two drugs replaced ChloroQuin (CQ) as a first-line treatment due to the rapid rise of CQ-resistant parasites. However, parasites resistant to SP evolved rapidly and now occur at high frequency in major malaria regions.

Studies have demonstrated that the rate of spontaneous mutation from drug sensitive to resistant alleles is about 10⁻⁸ per replication. If a typical infected host carries 10¹⁰ parasites in the body, at least 100 of them will be drug-resistant, and the number will keep increasing due to the selective advantage of resistant parasites over sensitive parasites within drug-treated hosts. Therefore, new resistant parasite strains would emerge readily whenever a drug is introduced to an endemic region.

If an infected person is untreated, drug-sensitive parasites will out-compete drug-resistant parasites and form the most prevalent population. After a mosquito infects a host, a typical dosage of anti-malarial drugs will likely kill most of the drug resistant parasites. However, the drug concentration decays over time. At an intermediate concentration, drug resistant parasites will increase their absolute numbers and resistant sporozoites will be further transmitted. If drugs decay slowly as most chemotherapeutic malaria medicines do, hosts with intermediate concentration are more prevalent, and resistant parasites have more opportunities to establish themselves. Limiting contact between infected hosts and mosquitoes while the drug decays over weeks would delay the spread of resistant parasites; however, this protection against a second infection and spread of resistant parasites requires hospitalization and mosquito netting to protect the patient, making the cost of treatment significantly higher.

2. Artemisinin

Although preparations of A. annua were used to treat malaria patients in ancient China, Artemisinin was not identified and extracted until the 1970s. A number of semi-synthetic Artemisinin derivatives with anti-malarial properties have been identified including artesunate, artemether, arteether, dihydroArtemisinin, artelinic acid, and artemotil. Artemisinin and its derivatives are now standard components of combination drug therapies to treat malaria. The principal reason that Artemisinin monotherapy is not in use is because of its poor water solubility, and hence its poor oral bioavailability. Artemisinin has a very short half-life resulting in rapid elimination. Therefore, Artemisinin treatment may not sustain plasma concentrations high enough to ensure complete elimination of blood parasites over several asexual cycles.

Metabolic and pharmacokinetic studies of Artemisinin derivatives such as artesunate, artemether, and arteether, show that they have a short half-life (1-2 h) in the blood when taken orally but have a longer half-life (7-9 h) when taken intramuscularly. Malaria infection itself has a significant effect on the pharmacokinetics of Artemisinin derivatives. For example, when artesunate was given orally, peak plasma concentrations and the relative bioavailability of dihydroArtemisinin (the major metabolite of Artemisinin derivatives) were higher in malaria patients given artesunate than in healthy patients [22]. Pharmacokinetic parameters also changed significantly for artesunate and dihydroArtemisinin in rats infected with Fasciola hepatica compared to healthy animals [23]. Bioavailability of Artemisinin derivatives may also change under other disease conditions such as cancer or malaria.

Artemisinin has generally not been used in mainstream clinical practice due to its poor bioavailability when compared to its derivatives and analogs. To overcome this limitation, current malaria treatments rely on ACT which combines the short-term effects of Artemisinin compounds with drugs that have a longer half-life due to lipid solubility in the patient and will kill the blood parasites over several asexual cycles. The current belief underlying ACT therapies is that Artemisinin will have a better effect if taken for a short period because, after 3-7 days of treatment, pharmacological levels of Artemisinin in the blood would decrease significantly due to degradation by induced CYP450 enzymes.

Artemisinin derivatives in the combinations include, but are not limited to, dihydroArtemisinin, artesunate and artemether. Companion drugs in the combination include, but are not limited to, lumefantrine, mefloquine, amodiaquine, sulfadoxine/pyrimethamine, piperaquine and chlorproguanil/dapsone. Unfortunately, ACT therapy also suffers from the problem of drug resistance because the companion drug stays in the body too long. Therefore, there is a repetitive cycle in ACT drug development where a particular anti-malaria drug will work for a time until resistance to that drug develops.

The following is an example of an ACT treatment protocol (Hombhanje & Huang, 2010) administering high doses of Artemisinin over a short period of time, which runs the danger of developing a parasite population that is resistant to both the chemotherapeutic malaria drug and Artemisinin. Although direct resistance to artemisinin or its derivatives is difficult to show, the evidence in using high dose vs. low dose (10%) cancer chemotherapeutic drugs is well established (43). Resistance has a better chance of developing if one gives relatively high dosages of Artemisinin rather than a low dose over a longer period of time.

Thirty-eight adult patients with symptomatic Plasmodium falciparum were treated orally under two dosing protocols. Patients were hospitalized for 6 days with three week follow-up. Protocol 1 administered 500 mg of Artemisinin×twice on day 1 (Total 1000 mg); and 250 mg Artemisinin×2 times daily (500 mg) on days 2-5 for a total dose of 3000 mg Artemisinin over 5 days. Protocol 2 administered 500 mg Artemisinin, then 750 mg of Artemisinin on Day 1 (total 1250 mg); and 250 mg×3 times (Total 750 mg) daily in co-administration with Melfloquine 250 mg×3 times (Total 750 mg) on Day 2 for a total dosage of 2000 mg Artemisinin over 2 days.

The use of high dose Artemisinin (up to 3000 mg) with ACT therapy significantly increases the possibility of developing a parasite resistant to Artemisinin. Analogies can be drawn from the bacteriology/antibiotic and cancer/chemotherapy worlds where more drug in a short timeframe is not always better.

There is a need for an alternative Artemisinin-based therapy which maximizes the therapeutic efficacy and minimizes the risk for development of resistance to Artemisinin.

### SUMMARY

The needs described above are addressed by the low-dose Artemisinin compound compositions and peroxybioflavonoid compositions provided in this disclosure as well as methods of treatment and/or prevention using such compositions. This disclosure describes a composition for treating and/or preventing a malaria infection in a patient in need of treatment or preventing, the compositions comprising a composition comprising, consisting of or consisting essentially of an Artemisinin compound or an Artemisinin compound and one or more bioflavonoid compounds (a peroxybioflavonoid composition). This disclosure also provides for methods of treatment and/or prevention of a malaria infection in a subject, the method comprising, consisting of or consisting essentially administering a composition comprising, consisting of or consisting essentially an Artemisinin compound or an Artemisinin compound and one or more bioflavonoid compounds (a peroxybioflavonoid composition).

The above summary presents a simplified summary in order to provide a basic understanding of some aspects of the claimed subject matter. This summary is not an extensive overview. It is not intended to identify key or critical elements or to delineate the scope of the claimed subject matter. Its sole purpose is to present some concepts in a simplified form as a prelude to the more detailed description that is presented later.

### BRIEF DESCRIPTION OF THE DRAWINGS

FIG. 1. Parasitic load over time in patients showing long-term clearance after peroxybioflavonoid treatment.

FIG. 2. Parasitic load over time in patients showing recurrence after peroxybioflavonoid treatment.

FIG. 3. Graphical representation of the parasitemia data from FIG. 1.

FIG. 4. Graphical representation of the parasitemia data from FIG. 2.

FIG. 5. Structure of liposomal NutraNanoSpheres for encapsulation of the Artemisinin/bioflavonoid combination treatment.

### DETAILED DESCRIPTION

### A Definitions

Unless otherwise defined, all terms (including technical and scientific terms) used herein have the same meaning as commonly understood by one of ordinary skill in the art of this disclosure. It will be further understood that terms, such as those defined in commonly used dictionaries, should be interpreted as having a meaning that is consistent with their meaning in the context of the specification and should not be interpreted in an idealized or overly formal sense unless expressly so defined herein. Well known functions or constructions may not be described in detail for brevity or clarity.

The terminology used herein is for the purpose of describing particular embodiments only and is not intended to be limiting. As used herein, the singular forms “a”, “an” and “the” are intended to include the plural forms as well, unless the context clearly indicates otherwise.

The terms “first”, “second”, and the like are used herein to describe various features or elements, but these features or elements should not be limited by these terms. These terms are only used to distinguish one feature or element from another feature or element. Thus, a first feature or element discussed below could be termed a second feature or element, and similarly, a second feature or element discussed below could be termed a first feature or element without departing from the teachings of the present disclosure.

The term “consisting essentially of” means that, in addition to the recited elements, what is claimed may also contain other elements (steps, structures, ingredients, components, etc.) that do not adversely affect the operability of what is claimed for its intended purpose as stated in this disclosure. Importantly, this term excludes such other elements that adversely affect the operability of what is claimed for its intended purpose as stated in this disclosure, even if such other elements might enhance the operability of what is claimed for some other purpose.

The terms “about” and “approximately” shall generally mean an acceptable degree of error or variation for the quantity measured given the nature or precision of the measurements. Typical, exemplary degrees of error or variation are within 20 percent (%), preferably within 10%, and more preferably within 5% of a given value or range of values. For biological systems, the term “about” refers to an acceptable standard deviation of error, preferably not more than 2-fold of a given value. Numerical quantities given herein are approximate unless stated otherwise, meaning that the term “about” or “approximately” can be inferred when not expressly stated.

The terms “prevention”, “prevent”, “preventing”, “suppression”, “suppress” and “suppressing” as used herein refer to a course of action initiated prior to the onset of a clinical manifestation of a disease state or condition so as to prevent or reduce such clinical manifestation of the disease state or condition. Such preventing and suppressing need not be absolute to be useful.

The terms “treatment”, “treat” and “treating” as used herein refers a course of action initiated after the onset of a clinical manifestation of a disease state or condition so as to eliminate or reduce such clinical manifestation of the disease state or condition. Such treating need not be absolute to be useful. In a particular aspect, the terms mean treating the subject in such a manner that recrudescence or the rate of recrudescence is decreased or eliminated.

The term “in need of treatment” as used herein refers to a judgment made by a caregiver that a patient requires or will benefit from treatment. This judgment is made based on a variety of factors that are in the realm of a caregiver's expertise, but that includes the knowledge that the patient is ill, or will be ill, as the result of a condition that is treatable by a method or device of the present disclosure.

The term “individual”, “subject” or “patient” as used herein refers to any animal, including mammals, such as mice, rats, other rodents, rabbits, dogs, cats, swine, cattle, sheep, horses, or primates, and humans. The term may specify male or female or both, or exclude male or female.

The term “therapeutically effective amount” as used herein refers to an amount of a compound, either alone or as a part of a supplement composition, that is capable of having any detectable, positive effect on any symptom, aspect, or characteristics of a disease state or condition. Such effect need not be absolute to be beneficial.

The term “prodrug” as used herein includes functional derivatives of a disclosed compound which are readily convertible in vivo into the required compound. Thus, in the methods of treatment of the present disclosure, the term “administering” shall encompass the treatment of the various disease states/conditions described with the compound specifically disclosed or with a prodrug which may not be specifically disclosed, but which converts to the specified compound in vivo after administration to the patient. Conventional procedures for the selection and preparation of suitable prodrug derivatives are described, for example, in “Design of Prodrugs”, ed. H. Bundgaard, Elsevier, 1985.

The term “physiologically acceptable salts” as used herein includes salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of physiologically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of physiologically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, oxalic, maleic, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The term “bioflavonoid” as used herein refers to compounds selected from the following classes: flavonones, flavonols, flavanones, flavans, flavanonols, anthocyanidins, proanthocyanidins isoflavones, isoflavans, isoflavandiols, isoflavenes, coumestans, pterocarpans, neoflavones and neoflavenes as well as stilbenes and stilbenoids. Specific subsets of bioflavonoids may be specified and preferred in certain combinations. In one aspect, the bioflavonoid is a Citrus bioflavonoid such as, but not limited to, rutin, quercitrin, hesperidin, naringin, hydroxyethylrutosides and combinations of the foregoing. In one aspect, the bioflavonoid is an anthocyanidin, such as, but not limited to, catechin, epicatechin, epicatechin gallate, epigallocatechin gallate, proanthocyanidins (oligomers of anthocyanidins) and combinations of the foregoing.

The term Artemisinin refers to the compound having the structure shown below, whether purified from a natural source or produced synthetically (either through complete synthesis or starting from a naturally derived intermediate) as well as physiologically acceptable salts and prodrugs thereof.

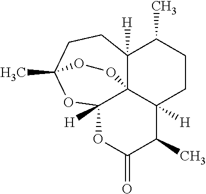

The term “Artemisinin derivative,” or “Artemisinin-derived” as used herein refers to purified plant-derived Artemisinin, semi-synthetic Artemisinin, and Artemisinin analogs and derivatives having a sesquiterpene lactone structure with a peroxide bridge, whether natural or semi-synthetic as well as physiologically acceptable salts and prodrugs thereof, including without limitation Artemether, Artesunate, Artenimol, Arteether, and DihydroArtemisinin.

The term “Artemisinin compound” as used herein refers to Artemisinin, an Artemisinin derivative or a combination of the foregoing. The specification may refer to a specific Artemisinin compound (for example, Artemisinin or DihydroArtemisinin) in certain embodiments discussed herein.

The term “peroxybioflavonoid” or “peroxybioflavonoid composition” refers to any combination of an Artemisinin compound and one or more bioflavonoids as described herein, in whatever dosing form may be utilized for delivery to a patient in need of treatment. In certain embodiments, the Artemisinin compound present in the peroxybioflavonoid composition is Artemisinin. In certain embodiments, the Artemisinin compound present in the peroxybioflavonoid composition is Artemisinin-derivative. In certain embodiments, the Artemisinin compound present in the peroxybioflavonoid composition is Artemether, Artesunate, Artenimol, Arteether, DihydroArtemisinin or combinations of the foregoing.

### B. Artemisinin-Containing Peroxybioflavonoid Compositions

A composition for increasing the bioavailability and efficacy of an Artemisinin compound for malaria treatment and/or prevention is provided which comprises: an Artemisinin compound (including Artemisinin or an Artemisinin derivative), including those extracted or concentrated from a natural source or synthesized from Artemisinin or an Artemisinin precursor, and a bioflavonoid compound, including those extracted or concentrated from a natural source, wherein the bioflavonoid compound is present in an amount effective to increase the bioavailability or anti-malarial effects of the Artemisinin compound.

1. Bioflavonoids

There are a number of ways to increase the bioavailability of an Artemisinin compound to act against erythrocytes and liver cells infected with Plasmodium sp. Bioflavonoids from A. annua or other biological sources may enhance the efficacy of Artemisinin compound therapy in the following ways: (1) they may inhibit the enzyme degradation of the Artemisinin compound; (2) they may function synergistically with the Artemisinin compound to kill the parasites; and (3) they may act as antioxidants and modulate the immune system.

A bioflavonoid-rich (plant-based) diet can play an important role in highly endemic malaria regions. It is known that morbidity caused by malaria is higher in children under the age of five and pregnant women who have a compromised immune system due to malnourishment. Thus, providing proper nutrition and increasing antioxidant levels through diet would have a pharmacological value to these afflicted populations. In addition, because antioxidant bioflavonoids are associated with immune system modulation, children receiving a healthy level of bioflavonoids in their diet could have a better chance to cope with and overcome P. falciparum infections.

Phenolic phytochemicals (phenolics), including bioflavonoids, occupy a unique position in the area of natural products due to their ubiquitous distribution throughout the plant kingdom and in products (fruits, vegetables, beverages, herbs, cosmetics and nutraceuticals) consumed by the general population on a regular basis [5]. Phenolics are biosynthesized by plants during normal development and in response to stress conditions such as exposure to UV radiation, pest attack, and wounding [6, 7]. Phenolic compounds are known to provide protection against a wide range of diseases such as coronary heart disease, stroke, and certain types of cancers [8, 9]. Chemically, phenolics are defined as a class of aromatic organic compounds with at least one hydroxyl group attached directly to a benzene ring [10]. Over 8000 phenolics with wide structural diversity and polarities have been isolated from plants [11]. For example, Chrysosplenol-D, quercetin-glucoside, flaviolin, rhamnetin, and pilloin were also reported as major leaf flavonoids of Artemisia annua [12].

For bioflavonoids to exert their role as oxidative stress modulators, antimalarial, anticancer, or synergistic effects, they are required to be absorbed from the gastrointestinal tract. Bioflavonoids show excellent absorption in the intestines, leading to their potential antioxidant effects in the blood [16]. The large pool of bioflavonoid derivatives resulting from microbial and mammalian metabolism might be responsible for their antioxidant activity [17, 18].

In general, plasma concentrations have been reported to vary from 0.3-0.75 μmoles/L after consumption of 800-100 mg of quercetin equivalents present in apples, onions, or meals rich in plant products. Values can be as high as 6.0 μmoles/L after consumption of 200 mg of naringenin, which is present in grapefruit juice [19]. There are two excellent reviews on bioavailability of polyphenols in humans [20, 21].

In contrast to the high-dose, short time period used in ACT, as described more fully below, therapy with low dose Artemisinin compounds and bioflavonoids may be provided in an oral dose using selective pH capsules that do not dissolve at pH 2 (stomach) where degradation of the Artemisinin compound would occur, but instead breakdown in the lower gut at pH 7. This delivery mechanism may result in preferential release of the compounds into the lower gastrointestinal tract rather than the stomach, thus resulting in higher bioavailability. Additionally, the use of liposomal encapsulation is an alternative embodiment that may enable the Artemisinin compound to pass directly into the bloodstream for increased bioavailability.

3. Effects of Bioflavonoids on Artemisinin

The bioflavonoids present in A. annua leaves have been linked to suppression of CYP450 enzymes responsible for altering the absorption and metabolism of Artemisinin compounds in the body, and also have been linked to a beneficial immunomodulatory activity in subjects afflicted with parasitic diseases. Additionally, it is known that bioflavonoids chelate metals such as iron and copper as part of their antioxidant effects and that iron chelating therapies have been recommended for malaria patients [2]. Bioflavonoids could be synergistic with Artemisinin compounds by reacting with iron and converting Fe⁺³ to Fe⁺² [3], the latter being important in the bioactivity of Artemisinin [4], leading to the release of short-lived toxic free radicals that are part of the antimalarial mode of action of Artemisinin. Thus, the use of bioflavonoids in combination with Artemisinin compounds, might provide a more effective treatment for malaria.

Citrus bioflavonoids, for example from grapefruit juice, significantly increased the oral bioavailability of Artemisinin without an effect on the elimination half-life [30]. There may be a possible role of intestinal CYP3A4, CYP450 and CYP2B5 (enzymes that play a role in degradation of Artemisinin) in the presystemic metabolism of Artemisinin [31]. Research has shown that grapefruit juice inhibits the action of Artemisinin-degrading enzymes with no inhibition of serum liver enzymes, when compared with uninfected controls. Co-administration of grapefruit juice with Artemisinin (150 mg/kg) completely protected mice from damage induced by Schistosoma mansoni infection, eliminated eggs, and prevented pathological granulomatous lesions [32].

The major constituents in grapefruit juice are bioflavonoids, of which the most prevalent is naringin, which is responsible for the bitter taste of grapefruit and is present at a concentration of up to 1,200 mg/mL. [33] The bioflavonoids of grapefruit juice have the ability to modulate CYP3A4 and p-glycoproteins, raising the potential of drug interactions and thus are considered to have synergistic affects with Artemisinin compounds. Because the effect of grapefruit juice can last up to 24 hours, repeated consumption can lead to a cumulative effect on the pharmacokinetics of co-administered drugs. Other herbs or herbal compounds that are known to inhibit CYP3A4 (intestinal) include bitter orange, berberine, and piperine [34]. Other bioflavonoids found in Citrus include apigenin, hesperidin, hesperitin, naringenin, narirutin, nobiletin, quercetin, rutin, and tangeretin.

Bioflavonoids also protect against oxidative stress through a mechanism that is not mediated by direct radical scavenging. For example, it has been shown that relatively low concentrations of bioflavonoids can increase intracellular glutathione levels via induction of transcription of gamma-glutamylcysteine synthetase [37]. Interestingly, this activity was selective for quercetin, kaempferol, and apigenin. Thus, it may well be that the pro-oxidant properties of some bioflavonoids may be instrumental for their final health beneficial properties, via interaction with transcriptional activities modulated by antioxidant-response/electrophile-response elements with the Nrf-2-KeaP-1 system as a major target [38]. Repeated mild cellular oxidative stress induced by bioflavonoids may boost cellular antioxidant defense systems and may cause long-term shifts in the defense system to a higher steady state, thereby preventing disease development or reducing the impact of oxidative stress when disease occurs [39].

1. Other Naturally Derived Compounds Synergistic with Artemisinin Compounds

Synergism with naturally derived compounds can improve the bioavailability of a drug with a wide safety margin such as Artemisinin compounds and lead to greater effectiveness of the treatment. For example, when the bioflavonoid curcumin was fed orally at 100 mg/kg to mice in combination with Artemisinin at 0.75 and 1.5 mg/mouse there was an increased survival rate for mice infected with Plasmodium berghei [24]. Although the dose of curcumin used was fairly high, those authors reported that curcumin toxicity is very low. It has been reported that Curcumin doses as high as 8.0 g/day were well tolerated by cancer patients for three months without toxicity.

Quercetin is wide spread in the plant kingdom and a major flavonoid of A. annua [25]. Quercetin was reported to increase the bioavailability of moxidectin (an anti-helmintic drug) in lambs [26].

The methoxylated flavonols chrysosplenol-D and chrysoplenetin, which alone had very weak growth inhibitory action, had a synergistic effect with berberine against Staphylococcus aureus [27]. The authors attributed this synergism to the inhibition of an S. aureus multidrug resistance (MDR) pump. Chrysosplenol-D, chrysoplenetin and other methoxylated flavonols produced by cell cultures of A. annua may potentiate the activity of Artemisinin against Plasmodium falciparum [28].

The bioflavonoids epigallocatechin gallate and catechin gallate were the most effective catechins from a crude green tea extract, found to have antimalarial effects of their own and to potentiate the effects of Artemisinin in vitro [29].

The danger of the continued use of high dose Artemisinin (1-3 grams) over short time periods (1-7 days) leads to the risk of creating wide-spread parasite resistant to Artemisinin, an important component of malaria treatment. Therefore, an effective, low-dose treatment protocol for Artemisinin compounds is needed to minimize the risk that resistance to Artemisinin compounds becomes widespread. Additionally, methods for enhancing the bioavailability and effectiveness of Artemisinin compounds in treatment and/or prevention are needed. This disclosure addresses these needs.

An inverse association between the development of lung cancer and the consumption of the highly-hydroxylated quercetin (from onion and apples) and naringin (from white grapefruit) by humans has been shown [35]. Methoxylated flavones have recently been shown to be more stable and present in much higher amounts than their hydroxylated counterparts in pharmacokinetic studies in rats using chrysin and its methoxylated version 5,7-dimethoxyflavone [36].

### Peroxybioflavonoid Compositions

It will be appreciated that each of the embodiments below is described in reference to certain preferred formulations. It will be appreciated that although each of the embodiments below is described in reference to an Artemisinin compound for simplicity in the description; the Artemisinin compound may be Artemisinin, an Artemisinin derivative or a combination of the foregoing. Suitable Artemisinin derivatives include, but are not limited to, Artemether, Artesunate, Artenimol, Arteether, and DihydroArtemisinin may be utilized in the disclosed compositions. For the sake of further clarity, each of the compositions described herein are referred to as a peroxybioflavonoid composition.

In one embodiment, the peroxybioflavonoid composition comprises, consists essentially or consists of an Artemisinin compound and one or more bioflavonoids.

In one embodiment, the peroxybioflavonoid composition comprises, consists essentially or consists of an Artemisinin compound and one or more bioflavonoids selected from the group consisting of: flavanols, flavonols, flavanones, flavones, isoflavones, and stillbenoids.

In one embodiment, the peroxybioflavonoid composition comprises, consists essentially or consists of an Artemisinin compound and one or more flavonols.

In one embodiment, the peroxybioflavonoid composition comprises, consists essentially or consists of an Artemisinin compound and one or more flavonols selected from the group consisting of: 3-hydroxyflavone, azaleatin, fisetin, galangin, gossypetin, kaempferide, kaempferol, isorhamnetin, morin, myricetin, natsudaidain, pachypodol, quercetin, rhamnazin, and rhamnetin.

In one embodiment, the peroxybioflavonoid composition comprises, consists essentially or consists of an Artemisinin compound and one or more flavanols.

In one embodiment, the peroxybioflavonoid composition comprises, consists essentially or consists of an Artemisinin compound and one or more flavanols selected from the group consisting of: catechin, epicatechin gallate, epigallocatechin, epigallocatechin gallate, proanthocyanidins, theaflavins, thearubigins.

In one embodiment, the peroxybioflavonoid composition comprises, consists essentially or consists of an Artemisinin compound and one or more flavanones.

In one embodiment, the peroxybioflavonoid composition comprises, consists essentially or consists of an Artemisinin compound and one or more flavanones selected from the group consisting of: butin, eriodictyol, hesperetin, hesperidin, homoeriodictyol, isosakuranetin, naringenin, naringin, pinocembrin, poncirin, sakuranetin, sakuranin, and sterubin.

In one embodiment, the peroxybioflavonoid composition comprises, consists essentially or consists of an Artemisinin compound and one or more flavones.

In one embodiment, the peroxybioflavonoid composition comprises, consists essentially or consists of an Artemisinin compound and one or more flavones selected from the group consisting of: apigenin, luteolin, tangeritin, chrysin, 6-hydroxyflavone, baicalein, scutellarein, wogonin, diosmin, flavoxate, and 7,8-dihydroxyflavone.

In one embodiment, the peroxybioflavonoid composition comprises, consists essentially or consists of an Artemisinin compound and one or more isoflavones.

In one embodiment, the peroxybioflavonoid composition comprises, consists essentially or consists of an Artemisinin compound and one or more isoflavones selected from the group consisting of: daidzein, biochanin-A, genistein, and glycitein.

In one embodiment, the peroxybioflavonoid composition comprises, consists essentially or consists of an Artemisinin compound and one or more stilbenoids

In one embodiment, the peroxybioflavonoid composition comprises, consists essentially or consists of an Artemisinin compound and one or more stilbenoids selected from the group consisting of: resveratrol and pterostilbene.

In one embodiment, the peroxybioflavonoid composition comprises, consists essentially or consists of an Artemisinin compound and one or more Citrus bioflavonoids.

In one embodiment, the peroxybioflavonoid composition comprises, consists essentially or consists of an Artemisinin compound and one or more Citrus bioflavonoids selected from the group consisting of: apigenin, hesperidin, hesperitin, naringenin, naringin, narirutin, nobiletin, quercetin, rutin, tangeretin, tangeritin.

In one embodiment, the peroxybioflavonoid composition comprises, consists essentially or consists of an Artemisinin compound and one or more Citrus bioflavonoids selected from the group consisting of: apigenin, hesperidin, hesperitin, naringenin, naringin, narirutin, nobiletin, quercetin, rutin, tangeretin, tangeritin, wherein hesperidin comprises at least 40% (such as at least 45%, at 50% or at least 55% or more) of the one or more Citrus bioflavonoids present in the composition (on a weight to weight basis of the total Citrus bioflavonoids present in the composition).

In one embodiment, the peroxybioflavonoid composition comprises, consists essentially or consists of an Artemisinin compound, hesperidin and optionally an additional bioflavonoids, wherein hesperidin comprises at least 40% (such as at least 45%, at 50% or at least 55% or more) of the total bioflavonoids present in the composition when additional bioflavonoids are present (on a weight to weight basis of the total bioflavonoids present in the composition).

In one embodiment, the peroxybioflavonoid composition comprises, consists essentially or consists of an Artemisinin compound and one or more bioflavonoids from A. annua.

In one embodiment, the peroxybioflavonoid composition comprises, consists essentially or consists of an Artemisinin compound and one or more bioflavonoids that increase the bioavailability of the Artemisinin compound when the Artemisinin compound is taken orally.

In one embodiment, the peroxybioflavonoid composition comprises, consists essentially or consists of an Artemisinin compound and one or more bioflavonoids with antioxidant properties.

In one embodiment, the peroxybioflavonoid composition comprises, consists essentially or consists of an Artemisinin compound and one or more bioflavonoids with anti-parasitic properties.

In one embodiment, the peroxybioflavonoid composition comprises, consists essentially or consists of an Artemisinin compound and one or more bioflavonoids with anti-bacterial properties.

In one embodiment, the peroxybioflavonoid composition comprises, consists essentially or consists of an Artemisinin compound and one or more bioflavonoids with anti-inflammatory properties.

In one embodiment, the peroxybioflavonoid composition comprises, consists essentially or consists of an Artemisinin compound and one or more bioflavonoids with anti-inflammatory properties selected from the group consisting of: casticin, chrysosplenol D, hesperidin.

In one embodiment, the peroxybioflavonoid composition comprises, consists essentially or consists of an Artemisinin compound and one or more bioflavonoids that affect the function of at least one of the group of enzymes selected from the group consisting of: CYP3A4, CYP450, and CYP2B5.

In one embodiment, the peroxybioflavonoid composition comprises, consists essentially or consists of an Artemisinin compound and one or more bioflavonoids that inhibit CYP3A4 (including intestinal CYP3A4).

In one embodiment, the peroxybioflavonoid composition comprises, consists essentially or consists of an Artemisinin compound and one or more bioflavonoids that inhibit CYP3A4 selected from the group consisting of: (including intestinal CYP3A4) including naringin, bitter orange, berberine, and piperine.

In certain aspects of the foregoing embodiments of the peroxybioflavonoid composition, when reference is made to an Artemisinin compound, the Artemisinin compound is Artemisinin. In certain aspects of the foregoing embodiments of the peroxybioflavonoid composition, when reference is made to an Artemisinin compound, the Artemisinin compound is an Artemisinin derivative. In certain aspects of the foregoing embodiments of the peroxybioflavonoid composition, when reference is made to an Artemisinin compound, the Artemisinin compound is an Artemisinin derivative selected from the group consisting of: Artemether, Artesunate, Artenimol, Arteether, and DihydroArtemisinin.

In certain aspects of the foregoing embodiments of the peroxybioflavonoid composition, when reference is made to an Artemisinin compound, the Artemisinin compound is Artemisinin and an Artemisinin derivative. In certain aspects of the foregoing embodiments of the peroxybioflavonoid composition, when reference is made to an Artemisinin compound, the Artemisinin compound is Artemisinin and an Artemisinin derivative selected from the group consisting of: Artemether, Artesunate, Artenimol, Arteether, and DihydroArtemisinin.

In one embodiment, the peroxybioflavonoid composition comprises, consists essentially or consists of Artemisinin and one or more Citrus bioflavonoids selected from the group consisting of: apigenin, hesperidin, hesperitin, naringenin, naringin, narirutin, nobiletin, quercetin, rutin, tangeretin, tangeritin, wherein hesperidin comprises at least 40% (such as at least 45%, at 50% or at least 55% or more) of the one or more Citrus bioflavonoids present in the composition (on a weight to weight basis of the total Citrus bioflavonoids present in the composition).

In one embodiment, the peroxybioflavonoid composition comprises, consists essentially or consists of Artemisinin, hesperidin and optionally an additional bioflavonoids, wherein hesperidin comprises at least 40% (such as at least 45%, at 50% or at least 55% or more) of the total bioflavonoids present in the composition when additional bioflavonoids are present (on a weight to weight basis of the total bioflavonoids present in the composition).

In one embodiment, the peroxybioflavonoid composition comprises, consists essentially or consists of at least one Artemisinin compound selected from the group consisting of Artemether, Artesunate, Artenimol, Arteether, and DihydroArtemisinin and one or more Citrus bioflavonoids selected from the group consisting of: apigenin, hesperidin, hesperitin, naringenin, naringin, narirutin, nobiletin, quercetin, rutin, tangeretin, tangeritin, wherein hesperidin comprises at least 40% (such as at least 45%, at 50% or at least 55% or more) of the one or more Citrus bioflavonoids present in the composition (on a weight to weight basis of the total Citrus bioflavonoids present in the composition).

In one embodiment, the peroxybioflavonoid composition comprises, consists essentially or consists of at least one Artemisinin compound selected from the group consisting of Artemether, Artesunate, Artenimol, Arteether, and DihydroArtemisinin, hesperidin and optionally an additional bioflavonoid, wherein hesperidin comprises at least 40% (such as at least 45%, at 50% or at least 55% or more) of the total bioflavonoids present in the composition when additional bioflavonoids are present (on a weight to weight basis of the total bioflavonoids present in the composition).

In one embodiment, the peroxybioflavonoid composition comprises, consists essentially or consists of Artemisinin, at least one Artemisinin compound selected from the group consisting of Artemether, Artesunate, Artenimol, Arteether, and DihydroArtemisinin and one or more Citrus bioflavonoids selected from the group consisting of: apigenin, hesperidin, hesperitin, naringenin, naringin, narirutin, nobiletin, quercetin, rutin, tangeretin, tangeritin, wherein hesperidin comprises at least 40% (such as at least 45%, at 50% or at least 55% or more) of the one or more Citrus bioflavonoids present in the composition (on a weight to weight basis of the total Citrus bioflavonoids present in the composition).

In one embodiment, the peroxybioflavonoid composition comprises, consists essentially or consists of Artemisinin, at least one Artemisinin compound selected from the group consisting of Artemether, Artesunate, Artenimol, Arteether, and DihydroArtemisinin, hesperidin and optionally an additional bioflavonoid, wherein hesperidin comprises at least 40% (such as at least 45%, at 50% or at least 55% or more) of the total bioflavonoids present in the composition when additional bioflavonoids are present (on a weight to weight basis of the total bioflavonoids present in the composition).

In certain aspects of the foregoing embodiments of the peroxybioflavonoid composition, the amount of the Artemisinin compound present in the composition is an amount up to about: 10 mg of Artemisinin, 15 mg of Artemisinin, 25 mg Artemisinin, 50 mg of Artemisinin, 75 mg of Artemisinin, 100 mg of Artemisinin, 150 mg of Artemisinin, 200 mg of Artemisinin, 300 mg of Artemisinin or a range between any two of the foregoing amounts. In certain aspects of the foregoing embodiments of the peroxybioflavonoid composition, the amount of the Artemisinin compound present in the composition is an amount between: about 10 mg and about 200 mg, about 15 and about 100 mg or about 20 mg and about 50 mg.

In certain aspects of the foregoing embodiments of the peroxybioflavonoid composition, the amount of the bioflavonoid (whether flavanols, flavonols, flavanones, flavones, isoflavones, stillbenoids or combinations thereof) is an amount up to about: 10 mg, 50 mg, 100 mg, 250 mg, 500 mg, 1000 mg, 1500 mg, 2000 mg, 2500 mg, 3000 mg, 3500 mg, 4000 mg, 4500 mg or 5000 mg. In certain aspects of the foregoing embodiments of the peroxybioflavonoid composition, the amount of the bioflavonoid (whether flavanols, flavonols, flavanones, flavones, isoflavones, stillbenoids or combinations thereof) present in the composition is an amount between: about 10 mg and about 5000 mg, about 10 mg and about 1000 mg, about 10 mg and about 500 mg, about 10 mg and about 200 mg, about 15 mg and about 100 mg or about 20 mg and about 50 mg.

In certain aspects of the foregoing embodiments of the peroxybioflavonoid composition, the amount of the Artemisinin compound present in the composition is an amount between: about 10 mg and about 200 mg, about 15 and about 100 mg or about 20 mg and about 50 mg and the amount of the bioflavonoid (whether flavanols, flavonols, flavanones, flavones, isoflavones, stillbenoids or combinations thereof) present in the composition is an amount between: about 10 mg and about 5000 mg, about 10 mg and about 1000 mg, about 10 mg and about 500 mg, about 10 mg and about 200 mg, about 15 mg and about 100 mg or about 20 mg and about 50 mg.

In certain aspects of the foregoing embodiments of the peroxybioflavonoid composition, the amount of the Artemisinin compound present in the composition is an amount between: about 10 mg and about 200 mg, about 15 and about 100 mg or about 20 mg and about 50 mg and the amount of the Citrus bioflavonoid present in the composition is an amount between: about 10 mg and about 5000 mg, about 10 mg and about 1000 mg, about 10 mg and about 500 mg, about 10 mg and about 200 mg, about 15 mg and about 100 mg or about 20 mg and about 50 mg.

In certain aspects of the foregoing embodiments of the peroxybioflavonoid composition, the amount of the Artemisinin compound present in the composition is an amount between: about 15 and about 100 mg or about 20 mg and about 50 mg and the amount of the Citrus bioflavonoid present in the composition is an amount between: about 15 mg and about 100 mg or about 20 mg and about 50 mg.

In certain aspects of the foregoing embodiments of the peroxybioflavonoid composition, the Artemisinin compound is Artemisinin and the amount of Artemisinin present in the composition is an amount between: about 10 mg and about 200 mg, about 15 and about 100 mg or about 20 mg and about 50 mg and bioflavonoid is hesperidin and the amount of hesperidin present in the composition is an amount between: about 10 mg and about 5000 mg, about 10 mg and about 1000 mg, about 10 mg and about 500 mg, about 10 mg and about 200 mg, about 15 mg and about 100 mg or about 20 mg and about 50 mg.

In certain aspects of the foregoing embodiments of the peroxybioflavonoid composition, the composition contains an Artemisinin compound and a bioflavonoid in a ratio of about 1:50, 1:25, 1:10, 1:5, 1:4 or 1:2, 1:1; 2:1, 4:1, 5:1; 10:1 (weight to weight, Artemisinin compound to bioflavonoid) or a range between any two of the foregoing ratios. In certain aspects of the foregoing embodiments of the peroxybioflavonoid composition, the composition contains an Artemisinin compound and a bioflavonoid in a ratio of about 2:1, 4:1, 5:1 or 10:1 (weight to weight, Artemisinin compound to bioflavonoid) or a range between any two of the foregoing ratios. In certain aspects of the foregoing embodiments of the peroxybioflavonoid composition, the composition contains an Artemisinin compound and a bioflavonoid in a ratio of about 1:1 (weight to weight, Artemisinin compound to bioflavonoid).

In certain aspects of the foregoing embodiments of the peroxybioflavonoid composition, the composition further comprises, consists of or consists essentially of an additional components, such as, but not limited to, vitamins and minerals. In certain aspects of the foregoing embodiments of the peroxybioflavonoid composition, the composition further comprises, consists of or consists essentially of a vitamin with antioxidant properties selected from the group consisting of: vitamin A, vitamin C and vitamin E. In certain aspects of the foregoing embodiments of the peroxybioflavonoid composition, the composition does not contain an additional component or drug that has been approved by a government agency for the treatment of malaria (with the proviso that a bioflavonoid is not included in this prohibition). In certain aspects of the foregoing embodiments of the peroxybioflavonoid composition, the composition does not contain chloroquine, quinine sulfate, hydroxychloroquine, mefloquine, atovaquone, proguanil, lumefantrine, doxycycline, primaquine, clindamycin, quinidine, pyrimethamine, amodiaquine, proguanil, sulfadoxine and sulfamethoxypyridazine, phenanthrene methano, amddiaquine, piperaquine chlorproguanil, dapsone and pyronaridine.

In certain aspects of the foregoing embodiments, the peroxybioflavonoid composition is provided in a single dosage form (i.e., each of the recited ingredients is in a single dosage form). hi certain aspects of the foregoing embodiments, the peroxybioflavonoid composition is provided in multiple dosage forms. For example, the Artemisinin compound is provided in a first dosage form and the bioflavonoid is provided in a second dosage form. When multiple dosage forms are provided for the peroxybioflavonoid composition, the dosage form for each component, may be the same or may be different. Suitable dosage forms include all dosage forms known in the art for administering Artemisinin compounds and bioflavonoids including, but not limited to, tablets, capsules, sachets, lozenges, troches, pills, powders, granules, elixirs, tinctures, solutions, suspensions, elixirs, and syrups as well as those described herein. In certain aspects of the foregoing embodiments, the peroxybioflavonoid composition is provided in a form for oral delivery. In certain aspects of the foregoing embodiments, the peroxybioflavonoid composition is provided in a form for transdermal delivery. In certain aspects of the foregoing embodiments, the peroxybioflavonoid composition is provided in a form for injection.

### C. Methods of Treatment

The present disclosure also provides for methods of treatment using the peroxybioflavonoid compositions described.

In one embodiment, the present disclosure provides a method of treating, suppressing or preventing a malaria infection in a subject, the method comprising the step of administering to the subject a peroxybioflavonoid composition as described herein.

In one embodiment, the present disclosure provides a method of treating, suppressing or preventing a malaria infection in a subject, the method comprising the step of administering to the subject a peroxybioflavonoid composition as described herein, wherein the peroxybioflavonoid composition is administered for at least 5 consecutive days (but less than 45 days).

In one embodiment, the present disclosure provides a method of treating, suppressing or preventing a malaria infection in a subject, the method comprising the step of administering to the subject a peroxybioflavonoid composition as described herein, wherein the peroxybioflavonoid composition is administered for at least 8 consecutive days (but less than 45 days).

In one embodiment, the present disclosure provides a method of treating, suppressing or preventing a malaria infection in a subject, the method comprising the step of administering to the subject a peroxybioflavonoid composition as described herein, wherein the peroxybioflavonoid composition is administered for at least 16 consecutive days (but less than 45 days).

In one embodiment, the present disclosure provides a method of treating, suppressing or preventing a malaria infection in a subject, the method comprising the step of administering to the subject a peroxybioflavonoid composition as described herein, wherein the peroxybioflavonoid composition is administered for at least 8 to 21 consecutive days (but less than 45 days).

In certain aspects of the foregoing methods, the total amount of the Artemisinin compound administered over the course of treatment is less than 2500 mg, less than 2000 mg, less than 1500 mg, less than 1000 mg, less than 500 mg or less than 300 mg but greater than 100 mg over the course of treatment. In certain aspects, the total amount of the Artemisinin compound administered over the course of treatment is between about 200 mg and about 1750 mg, such as 250 mg 400 mg, 800 mg, 1050 mg, or 1600 mg.

In certain aspects of the foregoing methods of treatment, the total amount of the bioflavonoid compound administered over the course of treatment is less than 5000 mg, less than 4000 mg, less than 2000 mg, less than 1000 mg, less than 500 mg, less than 250 mg, less than 100, less than 50 mg but greater than 10 mg. In certain aspects, the total amount of the bioflavonoid compound administered over the course of treatment is between about 50 mg and about 250 mg, such as 125 mg. In certain aspects, the total daily dose of the bioflavonoid compound administered is less than 200 mg, less than 150 mg, less than 100 mg, less than 50, less than 25 or less than or equal to 12.5 mg. In certain aspects, the total daily dose of the bioflavonoid compound administered is 25 mg. In certain aspects, the total daily dose of the bioflavonoid compound administered is 12.5 mg.

In one embodiment, the present disclosure provides a method of treating, suppressing or preventing a malaria infection in a subject, the method comprising the step of administering to the subject a peroxybioflavonoid composition as described herein, wherein the total amount of the Artemisinin compound administered is less than 2500 mg over the course of treatment but greater than 100 mg.

In one embodiment, the present disclosure provides a method of treating, suppressing or preventing a malaria infection in a subject, the method comprising the step of administering to the subject a peroxybioflavonoid composition as described herein, wherein the total amount of the Artemisinin compound administered is less than 2000 mg over the course of treatment but greater than 100 mg.

In one embodiment, the present disclosure provides a method of treating, suppressing or preventing a malaria infection in a subject, the method comprising the step of administering to the subject a peroxybioflavonoid composition as described herein, wherein the total amount of the Artemisinin compound administered is less than 1500 mg over the course of treatment but greater than 100 mg.

In one embodiment, the present disclosure provides a method of treating, suppressing or preventing a malaria infection in a subject, the method comprising the step of administering to the subject a peroxybioflavonoid composition as described herein, wherein the total amount of the Artemisinin compound administered is less than 1000 mg over the course of treatment but greater than 100 mg.

In one embodiment, the present disclosure provides a method of treating, suppressing or preventing a malaria infection in a subject, the method comprising the step of administering to the subject a peroxybioflavonoid composition as described herein, wherein the total amount of the Artemisinin compound administered is less than 500 mg over the course of treatment but greater than 100 mg.

In one embodiment, the present disclosure provides a method of treating, suppressing or preventing a malaria infection in a subject, the method comprising the step of administering to the subject a peroxybioflavonoid composition as described herein, wherein the total amount of the Artemisinin compound administered is less than 300 mg over the course of treatment but greater than 100 mg.

In one embodiment, the present disclosure provides a method of treating, suppressing or preventing a malaria infection in a subject, the method comprising the step of administering to the subject a peroxybioflavonoid composition as described herein, wherein the total amount of the Artemisinin compound administered is 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, or 1600 mg over the course of treatment.

In certain aspects of the foregoing methods of treatment, the total amount of the bioflavonoid compound administered over the course of treatment is less than 5000 mg, less than 4000 mg, less than 2000 mg, less than 1000 mg, less than 500 mg, less than 250 mg, less than 100, less than 50 mg but greater than 10 mg. In certain aspects, the total amount of the bioflavonoid compound administered over the course of treatment is between about 50 mg and about 250 mg, such as 125 mg. In certain aspects, the total daily dose of the bioflavonoid compound administered is less than 200 mg, less than 150 mg, less than 100 mg, less than 50 mg, less than 25 mg or less than or equal to 12.5 mg. In certain aspects, the total daily dose of the bioflavonoid compound administered is 25 mg. In certain aspects, the total daily dose of the bioflavonoid compound administered is 12.5 mg.

In one embodiment, the present disclosure provides a method of treating, suppressing or preventing a malaria infection in a subject, the method comprising the step of administering to the subject a peroxybioflavonoid composition as described herein, wherein the total daily dose of the Artemisinin compound administered does not exceed 250 mg per day.

In one embodiment, the present disclosure provides a method of treating, suppressing or preventing a malaria infection in a subject, the method comprising the step of administering to the subject a peroxybioflavonoid composition as described herein, wherein the total daily dose of the Artemisinin compound administered does not exceed 200 mg per day.

In one embodiment, the present disclosure provides a method of treating, suppressing or preventing a malaria infection in a subject, the method comprising the step of administering to the subject a peroxybioflavonoid composition as described herein, wherein the total daily dose of the Artemisinin compound administered does not exceed 150 mg per day.

In one embodiment, the present disclosure provides a method of treating, suppressing or preventing a malaria infection in a subject, the method comprising the step of administering to the subject a peroxybioflavonoid composition as described herein, wherein the total daily dose of the Artemisinin compound administered does not exceed 100 mg per day.

In one embodiment, the present disclosure provides a method of treating, suppressing or preventing a malaria infection in a subject, the method comprising the step of administering to the subject a peroxybioflavonoid composition as described herein, wherein the total daily dose of the Artemisinin compound administered does not exceed 50 mg per day.

In one embodiment, the present disclosure provides a method of treating, suppressing or preventing a malaria infection in a subject, the method comprising the step of administering to the subject a peroxybioflavonoid composition as described herein, wherein the total daily dose of the Artemisinin compound administered does not exceed 25 mg per day.

In certain aspects of the foregoing method, the total amount of the Artemisinin compound administered over the course of treatment is less than 2500 mg, less than 2000 mg, less than 1500 mg, less than 1000 mg, less than 500 mg or less than 300 mg but greater than 100 mg over the course of treatment. In certain aspects, the total amount of the Artemisinin compound administered over the course of treatment is between about 200 mg and about 1750 mg, such as 250 mg 400 mg, 800 mg, 1050 mg, or 1600 mg.

In certain aspects of the foregoing methods of treatment, the total amount of the bioflavonoid compound administered over the course of treatment is less than 5000 mg, less than 4000 mg, less than 2000 mg, less than 1000 mg, less than 500 mg, less than 250 mg, less than 100, less than 50 mg but greater than 10 mg. In certain aspects, the total amount of the bioflavonoid compound administered over the course of treatment is between about 50 mg and about 250 mg, such as 125 mg. In certain aspects, the total daily dose of the bioflavonoid compound administered is less than 200 mg, less than 150 mg, less than 100 mg, less than 50, less than 25 mg or less than or equal to 12.5 mg. In certain aspects, the total daily dose of the bioflavonoid compound administered is 25 mg. In certain aspects, the total daily dose of the bioflavonoid compound administered is 12.5 mg.

In one embodiment, the present disclosure provides a method of treating, suppressing or preventing a malaria infection in a subject, the method comprising the step of administering to the subject a peroxybioflavonoid composition as described herein, wherein the peroxybioflavonoid composition is administered for at least 5 consecutive days (but less than 45 days), the total amount of the Artemisinin compound administered is less than 2500 mg over the course of treatment and the total daily dose of the Artemisinin compound administered does not exceed 250 mg per day.

In certain aspects of the foregoing method, the total amount of the Artemisinin compound administered over the course of treatment is less than 2000 mg, less than 1500 mg, or less than 1000 mg but greater than 100 mg over the course of treatment. In certain aspects, the total amount of the Artemisinin compound administered over the course of treatment is between about 100 mg and about 1100 mg, such as 125 mg, 250 mg, 400 mg, 450 mg, 500 mg, 550 mg, 600 mg, 650 mg, 700 mg, 750 mg, 800 mg, 850 mg, 900 mg, 950 mg, 1000 mg or 1050 mg. In certain aspects, the total amount of the Artemisinin compound administered over the course of treatment is between about 100 mg and about 500 mg, such as 250 mg. In certain aspects, the total daily dose of the Artemisinin compound administered is less than 200 mg, less than 150 mg, less than 100 mg, less than or equal to 50 mg or less than or equal to 25 mg. In certain aspects, the total daily dose of the Artemisinin compound administered is 100 mg, 50 mg or 25 mg. In certain embodiments, a total daily dose of the Artemisinin compound is 100 mg or 50 mg when the subject is an adult or the subject is has a weight over 20 kg. In certain embodiments, a total daily dose of the Artemisinin compound is 50 mg or 25 mg when the subject has a weight less than or equal to 20 kg.

In certain aspects of the foregoing method, the total amount of the bioflavonoid compound administered over the course of treatment is less than 5000 mg, less than 4000 mg, less than 2000 mg, less than 1000 mg, less than 500 mg, less than 250 mg, less than 100, less than 50 mg but greater than 10 mg. In certain aspects, the total amount of the bioflavonoid compound administered over the course of treatment is between about 50 mg and about 250 mg, such as 125 mg. In certain aspects, the total daily dose of the bioflavonoid compound administered is less than 200 mg, less than 150 mg, less than 100 mg, less than 50, less than 25, or less than or equal to 12.5 mg. In certain aspects, the total daily dose of the bioflavonoid compound administered is 25 mg. In certain aspects, the total daily dose of the bioflavonoid compound administered is 12.5 mg.

In certain aspects of the foregoing method, the total amount of the Artemisinin compound administered over the course of treatment is between about 100 mg and about 500 mg, such as 250 mg, and the total amount of the bioflavonoid compound administered over the course of treatment is between about 50 mg and about 250 mg, such as 125 mg, wherein the total daily dose of the Artemisinin compound is less than 200 mg per day and the total daily dose of the bioflavonoid compound is less than 100 mg per day.

In one embodiment, the present disclosure provides a method of treating, suppressing or preventing a malaria infection in a subject, the method comprising the step of administering to the subject a peroxybioflavonoid composition as described herein, wherein the peroxybioflavonoid composition is administered for at least 8 consecutive days (but less than 45 days), the total amount of the Artemisinin compound administered is less than 2500 mg over the course of treatment and the total daily dose of the Artemisinin compound administered does not exceed 250 mg per day.

In certain aspects of the foregoing method, the total amount of the Artemisinin compound administered over the course of treatment is less than 2000 mg, less than 1500 mg, or less than 1000 mg but greater than 200 mg over the course of treatment. In certain aspects, the total amount of the Artemisinin compound administered over the course of treatment is between about 200 mg and about 2100 mg, such as 400 mg, 450 mg, 500 mg, 550 mg, 600 mg, 650 mg, 700 mg, 750 mg, 800 mg, 850 mg, 900 mg, 950 mg, 1000 mg, 1050 mg or 1600 mg. In certain aspects, the total amount of the Artemisinin compound administered over the course of treatment is between about 200 mg and about 800 mg, such as 400 mg. In certain aspects, the total daily dose of the Artemisinin compound administered is less than 200 mg, less than 150 mg, less than 100 mg, less than or equal to 50 mg or less than or equal to 25 mg. In certain aspects, the total daily dose of the Artemisinin compound administered is 100 mg, 50 mg or 25 mg. In certain embodiments, a total daily dose of the Artemisinin compound is 100 mg or 50 mg when the subject is an adult or the subject is has a weight over 20 kg. In certain embodiments, a total daily dose of the Artemisinin compound is 50 mg or 25 mg when the subject has a weight less than or equal to 20 kg.

In certain aspects of the foregoing method, the total amount of the bioflavonoid compound administered over the course of treatment is less than 5000 mg, less than 4000 mg, less than 2000 mg, less than 1000 mg, less than 500 mg, less than 250 mg, less than 100, less than 50 mg but greater than 10 mg. In certain aspects, the total amount of the bioflavonoid compound administered over the course of treatment is between about 75 mg and about 400 mg, such as 200 mg. In certain aspects, the total daily dose of the bioflavonoid compound administered is less than 200 mg, less than 150 mg, less than 100 mg, less than 50, less than 25, or less than or equal to 12.5 mg. In certain aspects, the total daily dose of the bioflavonoid compound administered is 25 mg. In certain aspects, the total daily dose of the bioflavonoid compound administered is 12.5 mg.

In certain aspects of the foregoing method, the total amount of the Artemisinin compound administered over the course of treatment is between about 200 mg and about 800 mg, such as 400 mg, and the total amount of the bioflavonoid compound administered over the course of treatment is between about 75 mg and about 400 mg, such as 200 mg, wherein the total daily dose of the Artemisinin compound is less than 200 mg per day and the total daily dose of the bioflavonoid compound is less than 100 mg per day.

In one embodiment, the present disclosure provides a method of treating, suppressing or preventing a malaria infection in a subject, the method comprising the step of administering to the subject a peroxybioflavonoid composition as described herein, wherein the peroxybioflavonoid composition is administered for at least 16 consecutive days (but less than 45 days), the total amount of the Artemisinin compound administered is less than 2500 mg over the course of treatment and the total daily dose of the Artemisinin compound administered does not exceed 250 mg per day.

In certain aspects of the foregoing method, the total amount of the Artemisinin compound administered over the course of treatment is less than 2000 mg, less than 1500 mg, or less than 1000 mg but greater than 200 mg over the course of treatment. In certain aspects, the total amount of the Artemisinin compound administered over the course of treatment is between about 200 mg and about 2100 mg, such as 400 mg, 450 mg, 500 mg, 550 mg, 600 mg, 650 mg, 700 mg, 750 mg, 800 mg, 850 mg, 900 mg, 950 mg, 1000 mg, 1050 mg or 1600 mg. In certain aspects, the total daily dose of the Artemisinin compound administered is less than 200 mg, less than 150 mg, less than 100 mg, less than or equal to 50 mg or less than or equal to 25 mg. In certain aspects, the total daily dose of the Artemisinin compound administered is 100 mg, 50 mg or 25 mg. In certain embodiments, a total daily dose of the Artemisinin compound is 100 mg or 50 mg when the subject is an adult or the subject is has a weight over 20 kg. In certain embodiments, a total daily dose of the Artemisinin compound is 50 mg or 25 mg when the subject has a weight less than or equal to 20 kg.

In certain aspects of the foregoing method, the total amount of the bioflavonoid compound administered over the course of treatment is less than 5000 mg, less than 4000 mg, less than 2000 mg, less than 1000 mg, less than 500 mg, less than 250 mg, less than 100, less than 50 mg but greater than 10 mg. In certain aspects, the total amount of the bioflavonoid compound administered over the course of treatment is between about 200 mg and about 800 mg, such as 400 mg. In certain aspects, the total daily dose of the bioflavonoid compound administered is less than 200 mg, less than 150 mg, less than 100 mg, less than 50, less than 25 mg or less than or equal to 12.5 mg. In certain aspects, the total daily dose of the bioflavonoid compound administered is 25 mg. In certain aspects, the total daily dose of the bioflavonoid compound administered is 12.5 mg.

In certain aspects of the foregoing method, the total amount of the Artemisinin compound administered over the course of treatment is between about 600 mg and about 1600 mg, such as 800 mg, and the total amount of the bioflavonoid compound administered over the course of treatment is between about 200 mg and about 800 mg, such as 400 mg, wherein the total daily dose of the Artemisinin compound is less than 200 mg per day and the total daily dose of the bioflavonoid compound is less than 100 mg per day.

In one embodiment, the present disclosure provides a method of treating, suppressing or preventing a malaria infection in a subject, the method comprising the step of administering to the subject a peroxybioflavonoid composition as described herein, wherein the peroxybioflavonoid composition is administered for 8 to 21 consecutive days, the total amount of the Artemisinin compound administered is less than 2500 mg over the course of treatment and the total daily dose of the Artemisinin compound administered does not exceed 250 mg per day.

In certain aspects of the foregoing method, the total amount of the Artemisinin compound administered over the course of treatment is less than 2000 mg, less than 1500 mg, or less than 1000 mg but greater than 200 mg over the course of treatment. In certain aspects, the total amount of the Artemisinin compound administered over the course of treatment is between about 200 mg and about 2100 mg, such as 400 mg, 450 mg, 500 mg, 550 mg, 600 mg, 650 mg, 700 mg, 750 mg, 800 mg, 850 mg, 900 mg, 950 mg, 1000 mg, 1050 mg or 1600 mg. In certain aspects, the total daily dose of the Artemisinin compound administered is less than 200 mg, less than 150 mg, less than 100 mg, less than or equal to 50 mg or less than or equal to 25 mg. In certain aspects, the total daily dose of the Artemisinin compound administered is 100 mg, 50 mg or 25 mg. In certain embodiments, a total daily dose of the Artemisinin compound is 100 mg or 50 mg when the subject is an adult or the subject is has a weight over 20 kg. In certain embodiments, a total daily dose of the Artemisinin compound is 50 mg or 25 mg when the subject has a weight less than or equal to 20 kg.

In certain aspects of the foregoing method, the total amount of the bioflavonoid compound administered over the course of treatment is less than 5000 mg, less than 4000 mg, less than 2000 mg, less than 1000 mg, less than 500 mg, less than 250 mg, less than 100, less than 50 mg but greater than 10 mg. In certain aspects, the total amount of the bioflavonoid compound administered over the course of treatment is between about 75 mg and about 1050 mg, such as 200 mg, 225 mg, 250 mg, 275 mg, 300 mg, 325 mg, 350 mg, 375 mg, 400 mg, 425 mg, 450 mg, 475 mg, 500 mg or 525 mg. In certain aspects, the total daily dose of the bioflavonoid compound administered is less than 200 mg, less than 150 mg, less than 100 mg, less than 50 or less than or equal to 25 mg. In certain aspects, the total daily dose of the bioflavonoid compound administered is 25 mg.

In certain aspects of the foregoing method, the total amount of the Artemisinin compound administered over the course of treatment is between about 200 mg and about 2100 mg, such as 400 mg, 450 mg, 500 mg, 550 mg, 600 mg, 650 mg, 700 mg, 750 mg, 800 mg, 850 mg, 900 mg, 950 mg, 1000 mg or 1050 mg, and the total amount of the bioflavonoid compound administered over the course of treatment is between about 75 mg and about 1050 mg, such as 200 mg, 225 mg, 250 mg, 275 mg, 300 mg, 325 mg, 350 mg, 375 mg, 400 mg, 425 mg, 450 mg, 475 mg, 500 mg or 525 mg, wherein the total daily dose of the Artemisinin compound is less than 200 mg per day and the total daily dose of the bioflavonoid compound is less than 100 mg per day.

In certain aspects of the foregoing embodiments, the subject is determined to be in need of treatment, in need of prevention or in need of suppression. Such determination may be made by a healthcare professional.

In certain aspects of the foregoing embodiments, the subject is a human. In certain aspects of the foregoing embodiments, the subject is under the age of 13 years. In certain aspects of the foregoing embodiments, the subject is over 13 years of age. In certain aspects of the foregoing embodiments, the subject is a male. In certain aspects of the foregoing embodiments, the subject is a female. In certain aspects of the foregoing embodiments, the subject has previously suffered from a malaria infection.

In certain aspects of the foregoing embodiments, the subject is infected with a Plasmodium species. In certain aspects of the foregoing embodiments, the subject is infected with P. falciparum, P malariae, P. ovale or P. vivax. In certain aspects of the foregoing embodiments, the subject is infected with one or more of P. falciparum, P malariae, P. ovale or P. vivax. In certain aspects of the foregoing embodiments, the subject is infected with a P. falciparum.

In certain aspects of the foregoing treatment embodiments, the peroxybioflavonoid composition is provided in a single dosage form (i.e., each of the recited ingredients is in a single dosage form). In certain aspects of the foregoing treatment embodiments, the peroxybioflavonoid composition is provided in multiple dosage forms. For example, the Artemisinin compound is provided in a first dosage form and the bioflavonoid is provided in a second dosage form. When multiple dosage forms are provided for the peroxybioflavonoid composition, the dosage form for each component may be the same or may be different. Suitable dosage forms include all dosage forms known in the art for administering Artemisinin compounds and bioflavonoids including, but not limited to, tablets, capsules, sachets, lozenges, troches, pills, powders, granules, elixirs, tinctures, solutions, suspensions, elixirs, and syrups as well as those described herein. In certain aspects of the foregoing embodiments, the peroxybioflavonoid composition is provided in a form for oral delivery. In certain aspects of the foregoing embodiments, the peroxybioflavonoid composition is provided in a form for transdermal delivery. In certain aspects of the foregoing embodiments, the peroxybioflavonoid composition is provided in a form for injection.

In certain aspects of the foregoing treatment embodiments, the peroxybioflavonoid composition is any of the peroxybioflavonoid compositions described herein.

In certain specific embodiments of the foregoing treatment embodiments, the peroxybioflavonoid composition is one of the following:

A peroxybioflavonoid composition comprising, consisting essentially of or consisting of an Artemisinin compound and one or more bioflavonoids.

A peroxybioflavonoid composition comprising, consisting essentially of or consisting of an Artemisinin compound and one or more Citrus bioflavonoids selected from the group consisting of: apigenin, hesperidin, hesperitin, naringenin, naringin, narirutin, nobiletin, quercetin, rutin, tangeretin, tangeritin.

A peroxybioflavonoid composition comprising, consisting essentially of or consisting of an Artemisinin compound and one or more Citrus bioflavonoids selected from the group consisting of: apigenin, hesperidin, hesperitin, naringenin, naringin, narirutin, nobiletin, quercetin, rutin, tangeretin, tangeritin, wherein hesperidin comprises at least 40% (such as at least 45%, at 50% or at least 55% or more) of the one or more Citrus bioflavonoids present in the composition (on a weight to weight basis of the total Citrus bioflavonoids present in the composition).

A peroxybioflavonoid composition comprising, consisting essentially of or consisting of an Artemisinin compound, hesperidin and optionally an additional bioflavonoids, wherein hesperidin comprises at least 40% (such as at least 45%, at 50% or at least 55% or more) of the total bioflavonoids present in the composition when additional bioflavonoids are present (on a weight to weight basis of the total bioflavonoids present in the composition).

In certain aspects of the foregoing peroxybioflavonoid compositions, when reference is made to an Artemisinin compound, the Artemisinin compound is Artemisinin. In certain aspects of the foregoing peroxybioflavonoid compositions, when reference is made to an Artemisinin compound, the Artemisinin compound is an Artemisinin derivative. In certain aspects of the foregoing peroxybioflavonoid compositions, when reference is made to an Artemisinin compound, the Artemisinin compound is an Artemisinin derivative selected from the group consisting of: Artemether, Artesunate, Artenimol, Arteether, and DihydroArtemisinin. In certain aspects of the foregoing peroxybioflavonoid compositions, when reference is made to an Artemisinin compound, the Artemisinin compound is Artemisinin and an Artemisinin derivative. In certain aspects of the foregoing peroxybioflavonoid compositions, when reference is made to an Artemisinin compound, the Artemisinin compound is Artemisinin and an Artemisinin derivative selected from the group consisting of: Artemether, Artesunate, Artenimol, Arteether, and DihydroArtemisinin.

In certain aspects the peroxybioflavonoid composition, the composition does not contain an additional component or drug that has been approved by a government agency for the treatment of malaria (with the proviso that a bioflavonoid is not included in this prohibition). In certain aspects of the peroxybioflavonoid composition, the composition does not contain chloroquine, quinine sulfate, hydroxychloroquine, mefloquine, atovaquone, proguanil, lumefantrine, doxycycline, primaquine, clindamycin, quinidine, pyrimethamine, amodiaquine, proguanil, sulfadoxine and sulfamethoxypyridazine, phenanthrene methano, amddiaquine, piperaquine chlorproguanil, dapsone and pyronaridine.

In certain specific embodiments of the foregoing treatment embodiments, the peroxybioflavonoid composition is one of the following:

A peroxybioflavonoid composition comprising, consisting essentially of or consisting of an Artemisinin compound and one or more bioflavonoids.

A peroxybioflavonoid composition comprising, consisting essentially of or consisting of Artemisinin and one or more bioflavonoids.

A peroxybioflavonoid composition comprising, consisting essentially of or consisting of at least one Artemisinin compound selected from the group consisting of Artemether, Artesunate, Artenimol, Arteether, and DihydroArtemisinin and one or more bioflavonoids.

A peroxybioflavonoid composition comprising, consisting essentially of or consisting of Artemisinin, at least one Artemisinin compound selected from the group consisting of Artemether, Artesunate, Artenimol, Arteether, and DihydroArtemisinin and one or more bioflavonoids.

A peroxybioflavonoid composition comprising, consisting essentially of or consisting of Artemisinin and one or more Citrus bioflavonoids selected from the group consisting of: apigenin, hesperidin, hesperitin, naringenin, naringin, narirutin, nobiletin, quercetin, rutin, tangeretin, tangeritin, wherein hesperidin comprises at least 40% (such as at least 45%, at 50% or at least 55% or more) of the one or more Citrus bioflavonoids present in the composition (on a weight to weight basis of the total Citrus bioflavonoids present in the composition).

A peroxybioflavonoid composition comprising, consisting essentially of or consisting of Artemisinin, hesperidin and optionally an additional bioflavonoids, wherein hesperidin comprises at least 40% (such as at least 45%, at 50% or at least 55% or more) of the total bioflavonoids present in the composition when additional bioflavonoids are present (on a weight to weight basis of the total bioflavonoids present in the composition).

A peroxybioflavonoid composition comprising, consisting essentially of or consisting of at least one Artemisinin compound selected from the group consisting of Artemether, Artesunate, Artenimol, Arteether, and DihydroArtemisinin and one or more Citrus bioflavonoids selected from the group consisting of: apigenin, hesperidin, hesperitin, naringenin, naringin, narirutin, nobiletin, quercetin, rutin, tangeretin, tangeritin, wherein hesperidin comprises at least 40% (such as at least 45%, at 50% or at least 55% or more) of the one or more Citrus bioflavonoids present in the composition (on a weight to weight basis of the total Citrus bioflavonoids present in the composition).

A peroxybioflavonoid composition comprising, consisting essentially of or consisting of at least one Artemisinin compound selected from the group consisting of Artemether, Artesunate, Artenimol, Arteether, and DihydroArtemisinin, hesperidin and optionally an additional bioflavonoids, wherein hesperidin comprises at least 40% (such as at least 45%, at 50% or at least 55% or more) of the total bioflavonoids present in the composition when additional bioflavonoids are present (on a weight to weight basis of the total bioflavonoids present in the composition).

A peroxybioflavonoid composition comprising, consisting essentially of or consisting of Artemisinin, at least one Artemisinin compound selected from the group consisting of Artemether, Artesunate, Artenimol, Arteether, and DihydroArtemisinin and one or more Citrus bioflavonoids selected from the group consisting of: apigenin, hesperidin, hesperitin, naringenin, naringin, narirutin, nobiletin, quercetin, rutin, tangeretin, tangeritin, wherein hesperidin comprises at least 40% (such as at least 45%, at 50% or at least 55% or more) of the one or more Citrus bioflavonoids present in the composition (on a weight to weight basis of the total Citrus bioflavonoids present in the composition).

A peroxybioflavonoid composition comprising, consisting essentially of or consisting of Artemisinin, at least one Artemisinin compound selected from the group consisting of Artemether, Artesunate, Artenimol, Arteether, and DihydroArtemisinin, hesperidin and optionally an additional bioflavonoids, wherein hesperidin comprises at least 40% (such as at least 45%, at 50% or at least 55% or more) of the total bioflavonoids present in the composition when additional bioflavonoids are present (on a weight to weight basis of the total bioflavonoids present in the composition).

In certain aspects the peroxybioflavonoid composition, the composition does not contain an additional component or drug that has been approved by a government agency for the treatment of malaria (with the proviso that a bioflavonoid is not included in this prohibition). In certain aspects of the peroxybioflavonoid composition, the composition does not contain chloroquine, quinine sulfate, hydroxychloroquine, mefloquine, atovaquone, proguanil, lumefantrine, doxycycline, primaquine, clindamycin, quinidine, pyrimethamine, amodiaquine, proguanil, sulfadoxine and sulfamethoxypyridazine, phenanthrene methano, amddiaquine, piperaquine chlorproguanil, dapsone and pyronaridine.

### D. Compositions Dosage Forms and Modes of Administration

The peroxybioflavonoid compositions of the present disclosure may be administered in any manner, including, but not limited to, orally, parenterally, sublingually, transdermally, vaginally, rectally, transmucosally, topically, via inhalation, via buccal or intranasal administration, or combinations thereof. Parenteral administration includes, but is not limited to, intravenous, intra-arterial, intra-peritoneal, subcutaneous, intramuscular, intra-thecal, and intra-articular. In a preferred embodiment, the peroxybioflavonoid compositions of the present disclosure are administered orally.

The dosage of the peroxybioflavonoid compositions of the present disclosure administered, as single or multiple doses, to an individual will vary depending upon a variety of factors, including pharmacokinetic properties, patient conditions and characteristics (sex, age, body weight, health, size), extent of symptoms, concurrent treatments, frequency of treatment and the effect desired. The daily dose ranges of the Artemisinin compound and bioflavonoid compound are provided herein.

The peroxybioflavonoid compositions of the present disclosure may contain an excipient. The term ‘excipient’ is defined generally as meaning a pharmacologically inactive substance formulated with the active pharmaceutical ingredient. The nature of the excipients varies depending on various factors such as the nature and concentration of the active ingredient, the subject to be treated, and the intended mode of administration.

The peroxybioflavonoid compositions of the present disclosure may take a number of forms. In one aspect the peroxybioflavonoid composition of the present disclosure is an oral composition. In one aspect the peroxybioflavonoid composition of the present disclosure is a liquid composition. In one aspect the peroxybioflavonoid composition of the present disclosure is a liquid oral composition (i.e. a liquid composition suitable for oral delivery, containing the active ingredients and excipients suitable for administration by ingestion). In this specification the term “liquid composition” includes any composition in which the active pharmaceutical ingredients are dispersed in a liquid matrix. The term “liquid” is defined according to its usual meaning in the art, i.e. the state of matter with a definite volume but no fixed shape and typically capable of flowing under pressure. It is therefore envisaged within the scope of the present invention that the term “liquid composition” includes suspensions, emulsions and solutions.

In one aspect the peroxybioflavonoid composition of the present disclosure is a solution. A solution is a homogeneous mixture composed of only one phase, in which the substance being dissolved (a solute) is dissolved in a liquid solvent. In particular, the peroxybioflavonoid composition of the present disclosure may be an oral solution (i.e. a solution containing the active ingredient and excipients suitable for administration by ingestion).

In one aspect the peroxybioflavonoid composition of the present disclosure is an emulsion. An emulsion is a mixture of two or more liquids that are normally immiscible, in which one liquid (the dispersed phase) is dispersed in the other (the continuous phase).

In one aspect the peroxybioflavonoid composition of the present disclosure is a suspension. A suspension is a heterogeneous mixture containing insoluble solid particles dispersed throughout a liquid (fluid), and in which the solid ultimately settles.

In one aspect the peroxybioflavonoid composition of the present disclosure is a solid oral dosage form. In one aspect, the solid oral dosage form comprises a shell or housing (typically of pharmaceutically acceptable materials as described and exemplified below) containing a liquid composition of the present disclosure (as described and exemplified herein, particularly in the form of a solution, suspension or emulsion).

In one aspect, the solid oral dosage form of the present invention is enteric coated. An enteric coating is a barrier applied to oral medication that controls the location in the digestive system where it is absorbed. Most enteric coatings work by presenting a surface that is stable at the highly acidic pH found in the stomach, but breaks down rapidly at a less acidic (relatively more basic) pH. For example, enteric coatings will not dissolve in the acidic juices of the stomach (pH ˜3), but will in the alkaline (pH 7-9) environment present in the small intestine. Materials used for enteric coatings include fatty acids, waxes, shellac, plastics, and plant fibres. Particular examples of materials used to form enteric coatings include methyl acrylate-methacrylic acid copolymers, cellulose acetate succinate, hydroxy propyl methyl cellulose phthalate, hydroxy propyl methyl cellulose acetate succinate (hypromellose acetate succinate), polyvinyl acetate phthalate (PVAP), methyl methacrylate-methacrylic acid copolymers, and sodium alginate and stearic acid.

In one aspect, the peroxybioflavonoid composition of the present disclosure is a capsule. Capsules are well known to those skilled in the art and typically comprise a shell capable of housing a liquid composition of the present disclosure (as described and exemplified herein, particularly in the form of a solution, suspension or emulsion), the shell comprising a material capable of being dissolved or degraded in vivo (in the gastrointestinal tract) to liberate the active ingredient. The capsules may be hard capsules (also known as hard-shelled capsules) or soft capsules (also called beads or soft-shelled capsules). The shells of both hard and soft of capsules are typically made from materials capable of being dissolved or degraded in vivo (in the gastrointestinal tract) to liberate the active ingredient. The material used to form the shell may be a gelling agent, in particular animal proteins such as gelatin; plant polysaccharides or their derivatives like carrageenans; natural or modified forms of starch and cellulose (in particular amylopectin, hydroxypropyl starch or hydroxypropylmethyl cellulose, HPMC); and polymers (in particular polyvinyl alcohol, PVA). Other ingredients can be added to the shell material solution like plasticizers such as glycerin and/or sorbitol to decrease the capsule's hardness, opacifying agents, flavourings, sweeteners, colouring agents, preservatives, disintegrants, lubricants and surface treatment.

In one aspect, the material used to form the shell comprises or consists of gelatin. Gelatin is a mixture of peptides and proteins typically produced by partial hydrolysis of collagen, a protein typically extracted from the skin, boiled crushed horn, hoof and bones, connective tissues, organs and some intestines of animals such as domesticated cattle, fish, chicken, pigs, and horses. The gelatin may also be modified as is known in the art to provide desired properties to the gelatin (for example, succinylated gelatin for use with reactive fill ingredients). In another aspect, the material used to form the shell comprises or consists of a material other than gelatin. Examples of hard capsules formed from non-gelatin shells include EcoCaps™ from Banner, the plant-derived VegiCaps™ from Catalent and Vegisoft™ from EuroCaps, and LiCaps™ from Capsugel. Examples of soft capsules formed from non-gelatin shells include Enteric softgels from Banner.

In one embodiment, the capsule is a hard capsule (especially a hard gelatin capsule). Typically,’ such hard capsules comprise empty upper and lower shells formed of rigid shell material. The formation of such capsules is a separate process and requires separate equipment from capsule filling. Typically, the bottom capsule shell is filled with the drug formulation, then the top capsule shell is placed over the lower filled shell. In one embodiment, the capsule is a soft capsule (especially a soft gelatin capsule). Typically, such a capsule takes the form of a single sealed flexible shell that contains the drug. In contrast to hard capsules, the process of forming soft capsules, comprising forming the shell and filling the shell with the drug formulation, can be achieved in a single process step. Typically this is carried out by forming sheets of the shell material, running the sheet through a mold to form a cavity, filling the cavity with the drug formulation, sealing the filled cavity, cutting/clipping the capsule from the gelatin ribbon, and drying.

In some embodiments, the peroxybioflavonoid composition comprises an Artemisinin compound and a bioflavonoid in combination or separately in a format suitable for oral delivery.

In some embodiments, the peroxybioflavonoid composition comprises an Artemisinin compound and a bioflavonoid in combination or separately in a format suitable for transdermal delivery, such as patches, creams, lotions, or pastes.

In some embodiments, the peroxybioflavonoid composition comprises an Artemisinin compound and a bioflavonoid in combination or separately in a format suitable for delivery by injection.

The peroxybioflavonoid combination may be prepared in the form of solid oral dosage form (for example, tablets or capsules) which are resistant to degradation at the pH environment of the stomach but readily dissolve in the pH environment of the intestine. This allows the Artemisinin compound and bioflavonoid to escape the degradative effects of the stomach and adds an additional protection of the Artemisinin compound and bioflavonoid so that maximum bioavailability is obtained.

Therefore, in some embodiments, the peroxybioflavonoid composition comprises an Artemisinin compound and a bioflavonoid in combination or separately in a dosing format that selectively dissolves at about pH 7 or in the pH environment of the intestine.

In some embodiments, the peroxybioflavonoid composition comprises an Artemisinin compound and a bioflavonoid in combination or separately in a dosing format that selectively dissolves in the pH environment of the intestine.

In some embodiments, the peroxybioflavonoid composition comprises an Artemisinin compound and a bioflavonoid in a capsule that selectively dissolves at about pH 7.

In some embodiments, the peroxybioflavonoid composition comprises a capsule of an Artemisinin compound that selectively dissolves at about pH 7 and a capsule of a bioflavonoid that selectively dissolves at about pH 7.

Liposomes are microscopic vesicles composed of a phospholipid bilayer that encapsulate active agents for specialized delivery to specific tissues. In certain embodiments, the peroxybioflavonoid compositions of the present disclosure are administered in a liposomal formulation.

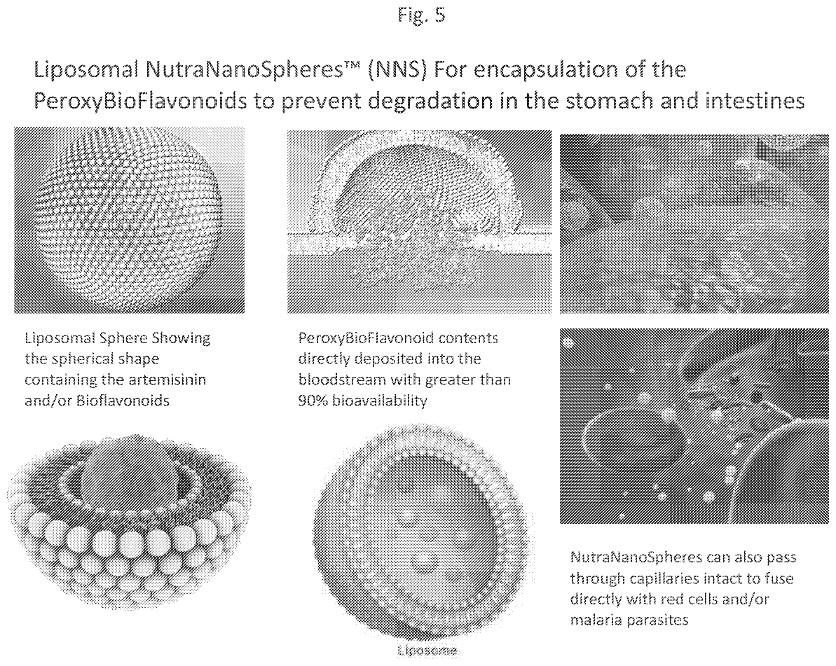

As shown in FIG. 5, the peroxybioflavonoid composition may also be encapsulated into a liposomal NutraNanoSphere. These drops that are accurately dispensed can be given orally without or without a drink, sublingually, through the skin, in a suppository, aerosol or as capsules where the drops are placed in the capsule. These methods of delivery of the liposomes allow the compounds to escape the degradative effects of the stomach and intestines and add an additional protection for the Artemisinin compound and bioflavonoid so that maximum bioavailability may be obtained. This combination is effective in patients treated for malaria in Nigeria and is near equivalent to the effectiveness of the standard capsule studies.

In some embodiments, the peroxybioflavonoid composition comprises an Artemisinin compound and a bioflavonoid in combination or separately in a format suitable for sublingual delivery.

In some embodiments, the peroxybioflavonoid composition comprises, consists essentially of or consists of an Artemisinin compound and bioflavonoid in a liposomal NutraNanoSphere. In some embodiments, the liposomal NutraNanoSphere contains about 50 mg of an Artemisinin compound and about 25 mg of bioflavonoid per two drops (100 ul). This allows babies (0-2 years) to be treated with one drop (25 mg Artemisinin compound/12.5 mg Bioflavonoid), children (2-12 years, under 44 Kg) to be treated with two drops (50 mg Artemisinin compound/25 mg Bioflavonoid), and adults (13 and up) to be treated with four drops (100 mg Artemisinin compound/50 mg Bioflavonoid).

In some embodiments, the liposomal NutraNanoSphere contains about 50 mg of Artemisinin compound and 25 mg of Citrus bioflavonoid per two drops (100 ul).

In some embodiments, the liposomal NutraNanoSphere contains about 100 mg of Artemisinin compound and 50 mg of Citrus bioflavonoid per two drops (100 ul).

In some embodiments, the liposomal NutraNanoSphere contains about 25 mg of Artemisinin compound and 25 mg of Citrus bioflavonoid per two drops (100 ul).

In some embodiments, the peroxybioflavonoid composition comprises Artemisinin compound, Citrus bioflavonoids, and curcumin.

In some embodiments, the peroxybioflavonoid composition comprises Artemisinin compound in a liposomal NutraNanoSphere and a bioflavonoid in a liposomal NutraNanoSphere.

In some embodiments, the peroxybioflavonoid composition further comprises curcumin in a liposomal NutraNanoSphere.

### E Examples

1. Clinical Trials in Haiti

In one example, patients in Haiti with non-complicated and complicated malaria (P. falciparum) infection were recruited to test the efficacy of peroxybioflavonoid treatment. The treated patient population included infants (n=7), children under age 18 (n>100), and adults (n>75). Prior to treatment, many patients would have two or more recurrences of malaria during a year despite taking chemotherapeutic pharmaceutical malaria medicines.

Each patient was given the peroxybioflavonoid composition over a 16 day period. Each day, babies were given 25 mg, children 2-12 years old received 50 mg and adults 100 mg of Artemisinin (low-dose Artemisinin). In some patients, Artemisinin was given alone. In other patients, the Artemisinin was combined with Citrus bioflavonoid (peroxybioflavonoid). Patients were treated for 16 days. After a single 16-day treatment, patients receiving the low-dose Artemisinin remained free of malaria for up to nine years post-treatment (n=17). According to estimates based upon studies in Africa, the expected rate of mosquito bites suffered by this Haitian patient population is 800-1,000 bites per year.

2. Clinical Trials in Nigeria

In another example, HIV-negative children between the ages 2-15 with P. falciparum load of 2,000-100,000 parasites per micro liter and a temperature between 37.5-38.4° C. were recruited. Those excluded from the clinical trial included patients with concomitant infection, patients treated with an anti-malarial drug in the week before presentation, and those with acute severe complicated malaria e.g. vomiting frequently that requires the administration of intravenous fluid, convulsion, severe anemia with PCV <18%, clinical evidence of pulmonary edema, feature suggestive of renal failure, history of Coca-Cola color urine which is suggestive of severe red blood cell hemolysis.

Samples were taken for thick blood film for malaria parasite on days 0, 1, 2, 3, 7, 14, 30 and 60; thin blood film for malaria parasite and PCV on days 0, 7, 14, 30 and 60; and IgM/IgG blood sample on day 0, 5, 10, 16, 30 and 60. Blood smears were air-dried, stained with a 2% Giemsa solution for 15 min, rinsed with water and re-air-dried and viewed under the microscope using oil immersion lens. Parasite densities were calculated from thick smears as the number of asexual parasites per 200 leukocytes (or per 500 leukocytes if the parasite density was <10 parasites per 200 leukocytes), assuming a leukocyte count of 6×10³ leukocytes/4 Smear findings were considered negative when microscopic examination of 100 high-power fields did not reveal parasites. Counts were performed by two microscopists and discrepant readings resolved by a third reader. Thin blood smears were performed to evaluate parasite species. Packed cell volumes were measured from finger-prick blood samples using heparinized capillary tube.

Patients were treated orally with a peroxybioflavonoid composition for 16 consecutive days. Each capsule contained 50 mg of Artemisinin (99% pure) and 25 mg Citrus Bioflavonoid (greater than 50% Hesperidin; in this example, 55.9%). Children over 12 years old or weighing more than 20 kg were administered 100 mg Artemisinin/day and 50 mg Citrus Bioflavonoid (two capsules) and children age two to 12 years old were administered 50 mg Artemisinin/day and 25 mg Citrus Bioflavonoid (one capsule). Of the 127 patients in the trial, 15 patients were lost to follow-up. Eleven failures were reported, three from one family suggesting non-compliance. The study shows that 90.2% of the patients showed no recurrence (termed “cured” in compliance with the literature definition where patients go at least 28 days without recurrence) by being parasite free by the standard blood tests at 30-60 days.

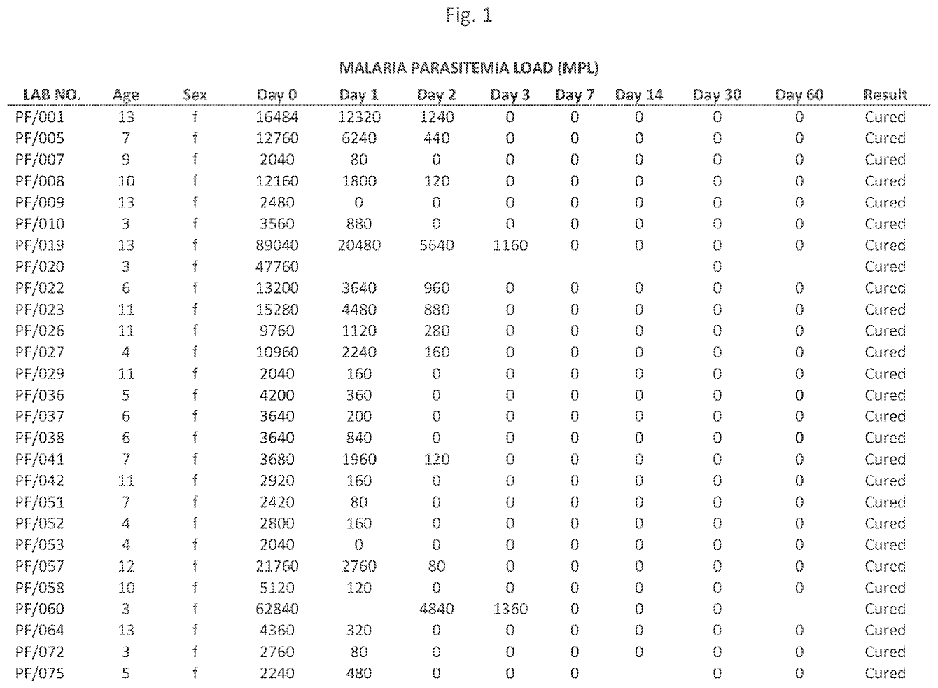

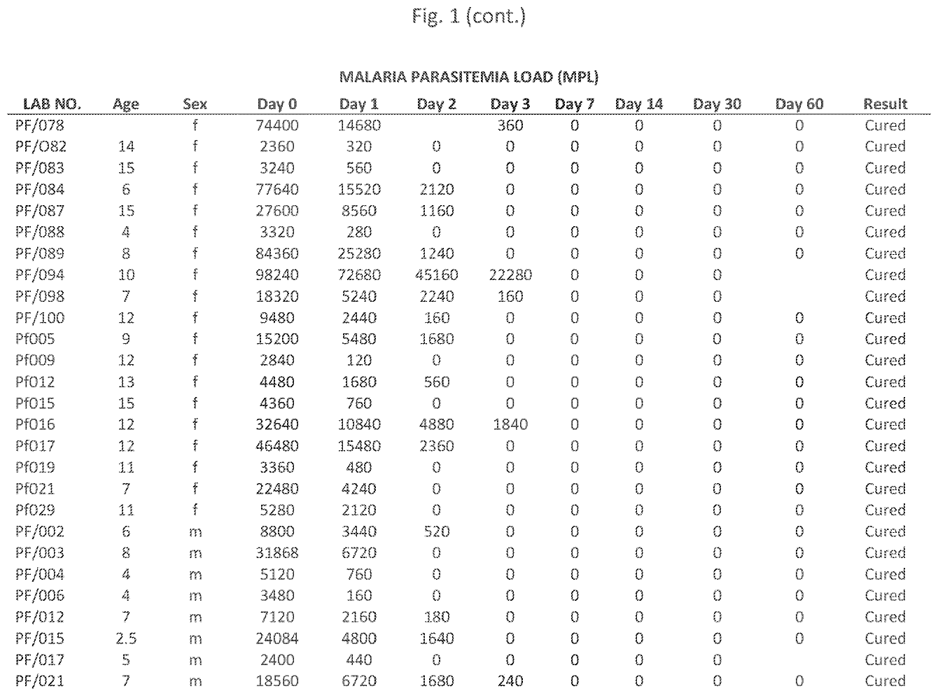

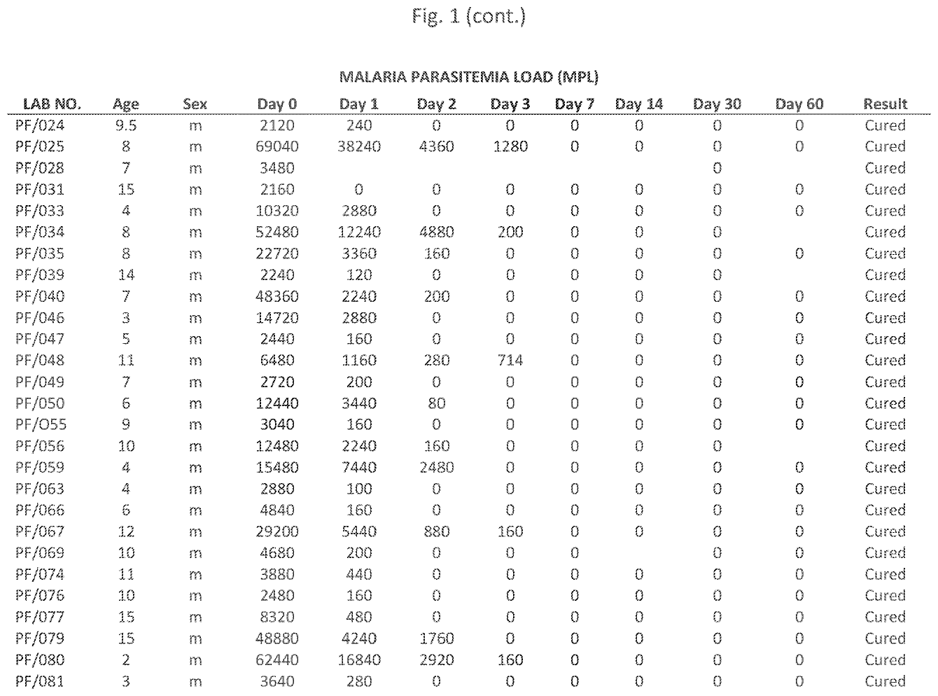

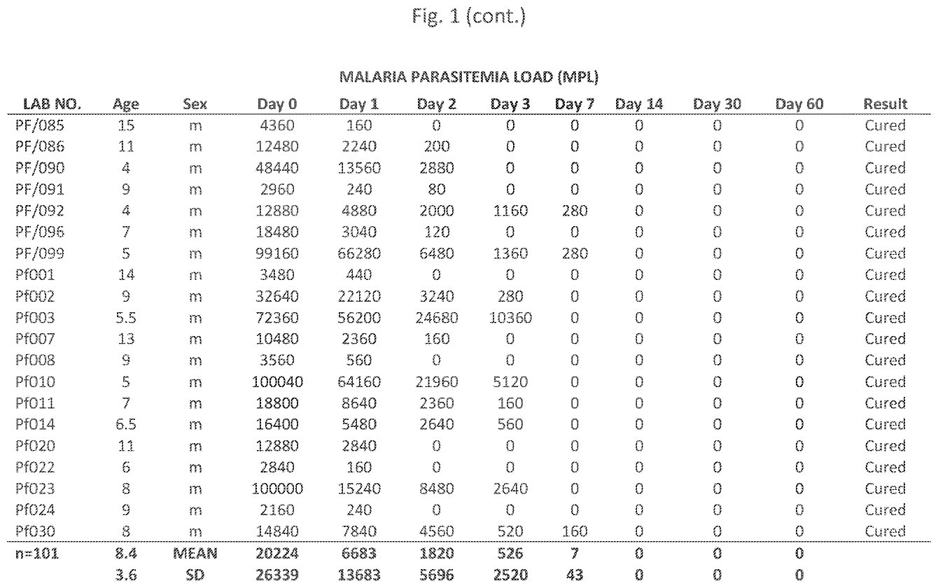

As shown in FIGS. 1-4, by day 3 of treatment almost all patients experienced a complete clearance of detectable parasite in blood smears. As shown in FIGS. 1 and 3, 92% (101/112) of the Nigerian patients with P. falciparum remained free of parasitic infection thirty to sixty days after the initiation of treatment.

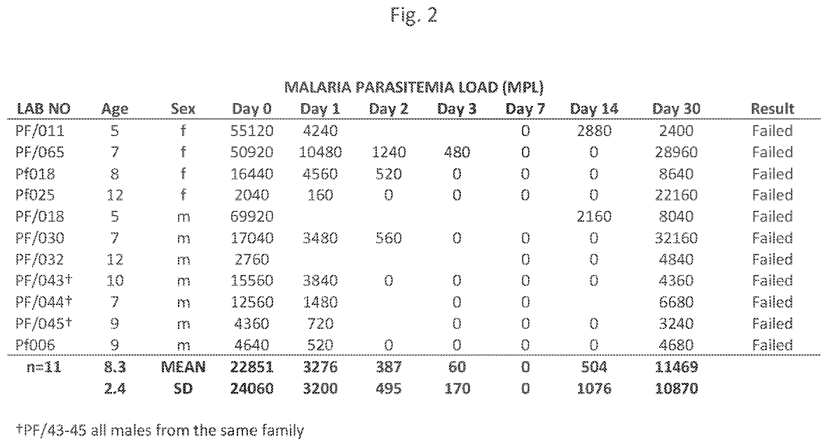

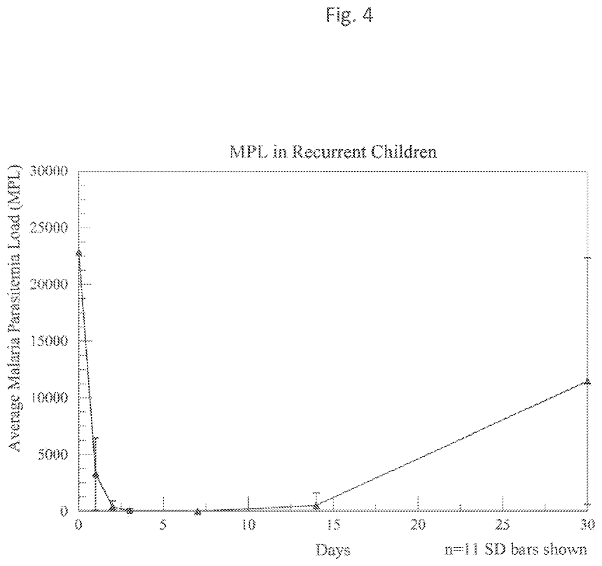

FIGS. 2 and 4 show that Nigerian patients experiencing a recurrence of P. falciparum infection showed a complete clearance of the parasites no later than day 7, with a rebound in parasite load first detectable at 14 days after the treatment was initiated.

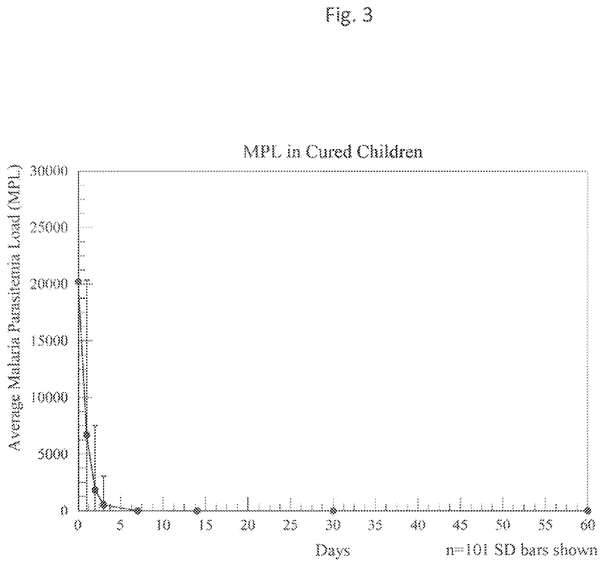

FIGS. 3 and 4 show there were no statistical differences in the average age or the initial parasitic load between those patients showing successful clearance or recurrence after treatment.

In some embodiments, a patient in need of treatment for malaria is administered peroxybioflavonoid composition as described herein one or more times per day for at least 8 or more days.

In some embodiments, a patient in need of treatment for malaria is administered peroxybioflavonoid composition as described herein one or more times per day for between 8 and 30 days.

In some embodiments, a patient in need of treatment for malaria is administered peroxybioflavonoid composition as described herein one or more times per day for between 14 and 21 days.

In some embodiments, a patient in need of treatment for malaria is administered peroxybioflavonoid composition as described herein one or more times per day for 16 days. Although the invention has been explained in relation to its preferred embodiment, it is to be understood that many other possible modifications and variations can be made without departing from the spirit and scope of the invention.

### REFERENCES

1. Stepniewska K, Taylor W R, Mayxay M, Price R, Smithuis F, et al. In vivo assessment of drug efficacy against Plasmodium falciparum malaria: duration of follow-up. Antimicrob Agents Chemother. 2004 November; 48(11):4271-80. PubMed PMID: 15504852; PubMed Central PMCID: PMC525402.
2. Wintola O A, Afolayan A J. The antibacterial, phytochemicals and antioxidants evaluation of the root extracts of Hydnora africanaThunb used as antidysenteric in Eastern Cape Province, South Africa. BMC Complement Altern Med. 2015 Sep. 4; 15:307. PubMed PMID: 26335685; PubMed Central PMCID: PMC4558922.
3. Gopalakrishnan A M, Kumar N. Antimalarial action of artesunate involves DNA damage mediated by reactive oxygen species. Antimicrob Agents Chemother. 2015 January; 59(1):317-25. PubMed PMID: 25348537; PubMed Central PMCID: PMC4291367.
4. Papanikolaou X, Johnson S, Garg T, Tian E, Tytarenko R, et al. Artesunate overcomes drug resistance in multiple myeloma by inducing mitochondrial stress and non-caspase apoptosis. Oncotarget. 2014 Jun. 30; 5(12):4118-28. PubMed PMID: 24948357; PubMed Central PMCID: PMC4147310.
5. Ribnicky D M, Roopchand D E, Poulev A, Kuhn P, Oren A, Cefalu W T, Raskin I. Artemisia dracunculus L. polyphenols complexed to soy protein show enhanced bioavailability and hypoglycemic activity in C57BL/6 mice. Nutrition. 2014 July-August; 30(7-8 Suppl):S4-10. doi: 10.1016/j.nut.2014.03.009. Epub 2014 Mar. 28. PubMed PMID: 24985105; PubMed Central PMCID: PMC4116120.
6. Tufts H R, Harris C S, Bukania Z N, Johns T. Antioxidant and Anti-Inflammatory Activities of Kenyan Leafy Green Vegetables, Wild Fruits, and Medicinal Plants with Potential Relevance for Kwashiorkor. Evid Based Complement Alternat Med. 2015; 2015:807158. PubMed PMID: 26236384; PubMed Central PMCID: PMC4510108.
7. Fernando C D, Soysa P. Extraction Kinetics of phytochemicals and antioxidant activity during black tea (Camellia sinensis L) brewing. Nutr J. 2015 Jul. 31; 14(1):74. PubMed PMID: 26226943; PubMed Central PMCID: PMC4520228.
8. Goldsmith C D, Vuong Q V, Sadeqzadeh E, Stathopoulos C E, Roach P D, et al. Phytochemical Properties and Anti-Proliferative Activity of Olea europaea L Leaf Extracts against Pancreatic Cancer Cells. Molecules. 2015 Jul. 17; 20(7):12992-3004. PubMed PMID: 26193251.
9. Kuete V, Efferth T. African flora has the potential to fight multidrug resistance of cancer. Biomed Res Int. 2015; 2015:914813. PubMed PMID: 25961047; PubMed Central PMCID: PMC4413252.
10. McCullough C, Neumann T S, Gone J R, He Z, Herrild C, et al. Probing the human estrogen receptor-.alpha. binding requirements for phenolic mono- and di-hydroxyl compounds: a combined synthesis, binding and docking study. Bioorg Med Chem. 2014 Jan. 1; 22(1):303-10. PubMed PMID: 24315190; NIHMSID: NIHMS548245; PubMed Central PMCID: PMC4013293.
11. Ntie-Kang F, Onguene P A, Lifongo L L, Ndom J C, Sippl W, et al. The potential of anti-malarial compounds derived from African medicinal plants, part II: a pharmacological evaluation of non-alkaloids and non-terpenoids. Malar J. 2014 Mar. 6; 13:81. PubMed PMID: 24602358; PubMed Central PMCID: PMC3975711.
12. Furst R, Zundorf I. Plant-derived anti-inflammatory compounds: hopes and disappointments regarding the translation of preclinical knowledge into clinical progress. Mediators Inflamm 2014; 2014:146832. PubMed PMID: 24987194; PubMed Central PMCID: PMC4060065.
13. Neto Z, Machado M, Lindeza A, do Rosario V, Gazarini M L, et al. Treatment of Plasmodium chabaudi Parasites with Curcumin in Combination with Antimalarial Drugs: Drug Interactions and Implications on the Ubiquitin/Proteasome System. J Parasitol Res. 2013; 2013:429736. PubMed PMID: 23691276; PubMed Central PMCID: PMC3649349.
14. Kumar Mishra S, Singh P, Rath S K. Protective effect of quercetin on chloroquine-induced oxidative stress and hepatotoxicity in mice. Malar Res Treat. 2013; 2013:141734. PubMed PMID: 23607047; PubMed Central PMCID: PMC3625570.
15. Fiorentini D, Zambonin L, Vieceli Dalla Sega F, Hrelia S. Polyphenols as Modulators of Aquaporin Family in Health and Disease. Oxid Med Cell Longev. 2015; 2015:196914. PubMed PMID: 26346093; PubMed Central PMCID: PMC4539495.
16. Marin L, Miguelez E M, Villar C J, Lombo F. Bioavailability of dietary polyphenols and gut microbiota metabolism: antimicrobial properties. Biomed Res Int. 2015; 2015:905215. PubMed PMID: 25802870; PubMed Central PMCID: PMC4352739.
17. Russo D, Valentao P, Andrade P B, Fernandez E C, Milella L. Evaluation of Antioxidant, Antidiabetic and Anticholinesterase Activities of Smallanthus sonchifolius Landraces and Correlation with Their Phytochemical Profiles. Int J Mol Sci. 2015 Jul. 31; 16(8):17696-718. PubMed PMID: 26263984.
18. Matsuoka M, Jin J. Application of Electrochemiluminescence for the Evaluation of the Antioxidant Capacity of Some Phenolic Compounds Against Superoxide Anion Radicals. Anal Sci. 2015; 31(7):629-34. PubMed PMID: 26165285.
19. Erlund I, Meririnne E, Alfthan G, Aro A. Plasma kinetics and urinary excretion of the flavanones naringenin and hesperetin in humans after ingestion of orange juice and grapefruit juice. J Nutr. 2001 February; 131(2):235-41. PubMed PMID: 11160539.
20. Upadhyay S, Dixit M. Role of Polyphenols and Other Phytochemicals on Molecular Signaling. Oxid Med Cell Longev. 2015; 2015:504253. PubMed PMID: 26180591; PubMed Central PMCID: PMC4477245.
21. Li A N, Li S, Zhang Y J, Xu X R, Chen Y M, et al. Resources and biological activities of natural polyphenols. Nutrients. 2014 Dec. 22; 6(12):6020-47. PubMed PMID: 25533011; PubMed Central PMCID: PMC4277013.
22. Zang M, Zhu F, Li X, Yang A, Xing J. Auto-induction of phase I and phase II metabolism of Artemisinin in healthy Chinese subjects after oral administration of a new Artemisinin-piperaquine fixed combination. Malar J. 2014 Jun. 3; 13:214. PubMed PMID: 24889062; PubMed Central PMCID: PMC4055232.
23. Zaloumis S G, Taming J, Krishna S, Price R N, White N J, et al. Population pharmacokinetics of intravenous artesunate: a pooled analysis of individual data from patients with severe malaria. CPT Pharmacometrics Syst Pharmacol. 2014 Nov. 5; 3:e145. PubMed PMID: 25372510; PubMed Central PMCID: PMC4259998.
24. Vathsala P G, Dende C, Nagaraj V A, Bhattacharya D, Das G, et al. Curcumin-arteether combination therapy of Plasmodium berghei-infected mice prevents recrudescence through immunomodulation. PLoS One. 2012; 7(1):e29442. PubMed PMID: 22276114; PubMed Central PMCID: PMC3262785.
25. Casas-Grajales S, Muriel P. Antioxidants in liver health. World J Gastrointest Pharmacol Ther. 2015 Aug. 6; 6(3):59-72. PubMed PMID: 26261734; PubMed Central PMCID: PMC4526841.
26. Dupuy J, Larrieu G, Sutra J F, Lespine A, Alvinerie M. Enhancement of moxidectin bioavailability in lamb by a natural flavonoid: quercetin. Vet Parasitol. 2003 Mar. 25; 112(4):337-47. PubMed PMID: 12623212.
27. Dziedzic A, Wojtyczka R D, Kubina R Inhibition of Oral Streptococci Growth Induced by the Complementary Action of Berberine Chloride and Antibacterial Compounds. Molecules. 2015 Jul. 28; 20(8):13705-24. PubMed PMID: 26225951.
28. Zhu X X, Yang L, Li Y J, Zhang D, Chen Y, et al. Effects of sesquiterpene, flavonoid and coumarin types of compounds from Artemisia annua L on production of mediators of angiogenesis. Pharmacol Rep. 2013; 65(2):410-20. PubMed PMID: 23744425.
29. Patil P R, Gemma S, Campiani G, Craig A G. Broad inhibition of Plasmodium falciparum cytoadherence by (+)-epigallocatechin gallate. Malar J. 2011 Dec. 1; 10:348. PubMed PMID: 22132804; PubMed Central PMCID: PMC3260111.
30. Chu Y, Wang H, Chen J, Hou Y. New Sesquiterpene and Polymethoxy-Flavonoids from Artemisia annua L. Pharmacogn Mag. 2014 July; 10(39):213-6. PubMed PMID: 25210306; PubMed Central PMCID: PMC4159912.
31. Xu L, Sun L, Lu L, Zhong X, Ma Y, et al. Effects of quercetin on CYP450 and cytokines in Aroclor 1254 injured endometrial cells of the pregnant rats. Biomed Res Int. 2014; 2014:497508. PubMed PMID: 24711995; PubMed Central PMCID: PMC3966338.
32. Zhang Z S, Wang J, Shen Y B, Guo C C, Sai K E, et al. DihydroArtemisinin increases temozolomide efficacy in glioma cells by inducing autophagy. Oncol Lett. 2015 July; 10(1):379-383. PubMed PMID: 26171034; PubMed Central PMCID: PMC4487108.
33. Wang B, Lin S Y, Shen Y Y, Wu L Q, Chen Z Z, et al. Pure total flavonoids from Citrus paradisi Macfadyen act synergistically with arsenic trioxide in inducing apoptosis of Kasumi-1 leukemia cells in vitro. J Zhejiang Univ Sci B. 2015 July; 16(7):580-5. PubMed PMID: 26160715; PubMed Central PMCID: PMC4506948.
34. Tanaka S, Uchida S, Miyakawa S, Inui N, Takeuchi K, et al. Comparison of inhibitory duration of grapefruit juice on organic anion-transporting polypeptide and cytochrome P450 3A4. Biol Pharm Bull. 2013; 36(12):1936-41. PubMed PMID: 24292052.
35. Woo H D, Kim J. Dietary flavonoid intake and smoking-related cancer risk: a meta-analysis. PLoS One. 2013; 8(9):e75604. PubMed PMID: 24069431; PubMed Central PMCID: PMC3777962.
36. Walle T. Methoxylated flavones, a superior cancer chemopreventive flavonoid subclass?. Semin Cancer Biol. 2007 October; 17(5):354-62. PubMed PMID: 17574860; NIHMSID: NIHMS31198; PubMed Central PMCID: PMC2024817.
37. Ayyathan D M, Chandrasekaran R, Thiagarajan K. Neuroprotective effect of Tagara, an Ayurvedic drug against methyl mercury induced oxidative stress using rat brain mitochondrial fractions. BMC Complement Altern Med. 2015 Aug. 12; 15:268. PubMed PMID: 26264039; PubMed Central PMCID: PMC4533944.
38. Chedea V S, Braicu C, Chiril F, Ogola H J, Pelmu R, et al. Antioxidant/Prooxidant and antibacterial/probacterial effects of a grape seed extract in complex with lipoxygenase. Biomed Res Int. 2014; 2014:313684. PubMed PMID: 25313359; PubMed Central PMCID: PMC4181939.
39. Martinez Mdel C, Afonso S G, Buzaleh A M, Baffle A. Protective action of antioxidants on hepatic damage induced by griseofulvin. ScientificWorldJournal. 2014; 2014:982358. PubMed PMID: 24523661; PubMed Central PMCID: PMC3913521.
40. Moreno-Garcia M, Vargas V, Ramirez-Bello I, Hernandez-Martinez G, Lanz-Mendoza H. Bacterial Exposure at the Larval Stage Induced Sexual Immune Dimorphism and Priming in Adult Aedes aegypti Mosquitoes. PLoS One. 2015; 10(7):e0133240. PubMed PMID: 26181517; PubMed Central PMCID: PMC4504673.
41. Bianchin A, Bell A, Chubb A J, Doolan N, Leneghan D, et al. Design and evaluation of antimalarial peptides derived from prediction of short linear motifs in proteins related to erythrocyte invasion. PLoS One. 2015; 10(6):e0127383. PubMed PMID: 26039561; PubMed Central PMCID: PMC4454681.
42. Angelique L, Frederik W J, Garmi J, Hester du P L. The Potential Use of Natural and Structural Analogues of Antimicrobial Peptides in the Fight against Neglected Tropical Diseases. Molecules. 2015 Aug. 24; 20(8):15392-433. PubMed PMID: 26305243.
43. Finn O J. Immuno-oncology: understanding the function and dysfunction of the immune system in cancer. Ann Oncol. 2012 September; 23 Suppl 8:viii6-9. PMID: 22918931

I claim:

1. A method for treating, suppressing, or preventing malaria in a subject in need thereof, the method comprising the step of administering to the subject a composition comprising an Artemisinin compound and one or more bioflavonoid compounds for at least 16 consecutive days.
